SEQUENCE LISTING (12 sequences)

SEQ ID NO: 1 - protein, 9 aa, Homo sapiens
ASIKVAVSA

SEQ ID NO: 2 - protein, 5 aa, Homo sapiens
DKPRR

SEQ ID NO: 3 - protein, 16 aa, synthetic construct
ASIKVAVSAGGDKPRR

SEQ ID NO: 4 - protein, 19 aa, Homo sapiens
CSRARKQAASIKVAVSADR

SEQ ID NO: 5 - protein, 6 aa, Homo sapiens
CDKPRR

SEQ ID NO: 6 - protein, 11 aa, synthetic construct
GASIKVAVSAG

SEQ ID NO: 7 - protein, 414 aa, synthetic construct
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHSPSCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 8 - DNA, 1245 bp, synthetic construct
  source 1..1245: mol_type = other DNA
atgcactactgtgtgctgagcgcttttctgatcctgcatctggtcacggtcgcgctcagcctgtctacctgcagcacactcgatatggaccagttcatgcgcaagaggatcgaggcgatccgcgggcagatcctgagcaagctgaagctcaccagtcccccagaagactatcctgagcccgaggaagtccccccggaggtgatttccatctacaacagcaccagggacttgctccaggagaaggcgagccggagggcggccgcctgcgagcgcgagaggagcgacgaagagtactacgccaaggaggtttacaaaatagacatgccgcccttcttcccctccgaaaatgccatcccgcccactttctacagaccctacttcagaattgttcgatttgacgtctcagcaatggagaagaatgcttccaatttggtgaaagcagagttcagagtctttcgtttgcagaacccaaaagccagagtgcctgaacaacggattgagctatatcagattctcaagtccaaagatttaacatctccaacccagcgctacatcgacagcaaagttgtgaaaacaagagcagaaggcgaatggctctccttcgatgtaactgatgctgttcatgaatggcttcaccataaagacaggaacctgggatttaaaataagcttacactctccctcttgcacttttgtaccatctaataattacatcatcccaaataaaagtgaagaactagaagcaagatttgcaggtattgatggcacctccacatataccagtggtgatcagaaaactataaagtccactaggaaaaaaaacagtgggaagaccccacatctcctgctaatgttattgccctcctacagacttgagtcacaacagaccaaccggcggaagaagcgtgctttggatgcggcctattgctttagaaatgtgcaggataattgctgcctacgtccactttacattgatttcaagagggatctagggtggaaatggatacacgaacccaaagggtacaatgccaacttctgtgctggagcatgcccgtatttatggagttcagacactcagcacagcagggtcctgagcttatataataccataaatccagaagcatctgcttctccttgctgcgtgtcccaagatttagaacctctaaccattctctactacattggcaaaacacccaagattgaacagctttctaatatgattgtaaagtcttgcaaatgcagctaa

SEQ ID NO: 9 - protein, 359 aa, Homo sapiens
MKATIILLLLAQVSWAGPFQQRGLFDFMLEDEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 10 - DNA, 1080 bp, synthetic construct
  source 1..1080: mol_type = other DNA
atgaaggccactatcatcctccttctgcttgcacaagtttcctgggctggaccgtttcaacagagaggcttatttgactttatgctagaagatgaggcttctgggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtaa

SEQ ID NO: 11 - DNA, 2211 bp, synthetic construct
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 12 - DNA, 2244 bp, synthetic construct
  source 1..2244: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaaggagccagtatcaaggtcgccgtgagtgccggagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

(19) United States
(12) Patent Application Publication
(10) Pub. No.: US 2025/0195692 A1
(43) Pub. Date: Jun. 19, 2025

(54) GENE THERAPY FOR GLAUCOMA

(71) Applicant: Trustees of Tufts College, Medford, MA (US)

(72) Inventor: Rajendra Kumar-Singh, Medford, MA (US)

(21) Appl. No.: 18/983,102

(22) Filed: Dec. 16, 2024

Related U.S. Application Data
(60) Provisional application No. 63/610,383, filed on Dec. 14, 2023.

(51) Int. Cl. A61K 48/00; A01K 67/0278; A61K 9/00; A61K 38/10; A61K 38/39; A61P 27/06; C07K 14/495; C12N 15/86

(52) U.S. Cl. CPC A61K 48/005; A01K 67/0278; A61K 9/0048; A61K 38/10; A61K 38/39; A61P 27/06; C07K 14/495; C12N 15/86; A01K 2217/052; A01K 2267/0306; C12N 2750/14122; C12N 2750/14143

(57) ABSTRACT

Disclosed are methods of treating an eye disease by administering a recombinant adeno-associated virus (AAV) vector that encodes the proteoglycan decorin to the eye of a subject. Also provided are animal models of glaucoma that are generated using a recombinant AAV vector that encodes a constitutively active variant of transforming growth factor beta 2 (TGFβ2^{CS}).

### CROSS-REFERENCE TO RELATED APPLICATIONS

This application claims priority to U.S. Provisional Application No. 63/610,383 filed on Dec. 14, 2023, the contents of which are incorporated by reference in their entireties.

### SEQUENCE LISTING STATEMENT

This application includes a sequence listing in XML format titled “166118_01473_ST26.xml”, which is 18,876 bytes in size and was created on Dec. 3, 2024. The sequence listing is electronically submitted with this application via Patent Center and is incorporated herein by reference in its entirety.

### BACKGROUND

Primary open angle glaucoma (POAG) is the most common cause of irreversible blindness, affecting approximately 76 million individuals globally (1). In addition, approximately 30,000 infants are born each year with cataract, and infantile aphakic glaucoma (IAG) following cataract surgery is considered the most sight-threatening complication of pediatric cataract care (2, 3).

The cardinal and irreversible features of glaucoma include the loss of retinal ganglion cells (RGCs) and their axons that form the optic nerve (4, 5). The major risk factor associated with glaucoma is elevated intra ocular pressure (IOP) (4, 5). The causal link between elevated IOP and loss of RGCs is not completely understood, but interventions such as drugs or surgery that alleviate IOP have been found to attenuate progression towards blindness (4, 5).

The ciliary body continuously produces aqueous humor, which exits the eye via the trabecular meshwork (4, 5). Accumulation of extracellular matrix deposits in the trabecular meshwork can impede drainage of aqueous humor, resulting in an increase in IOP (4, 5). Almost all drugs or surgical interventions for the treatment of glaucoma target either the production of aqueous humor by the ciliary body or enhance the flow of aqueous humor through the trabecular meshwork (6, 7). Despite the availability of such drugs, glaucoma continues to be a significant cause of visual impairment. Thus, there is an unmet clinical need for improved strategies to regulate IOP in glaucoma.

Genetic studies of glaucoma have revealed that it is a complex and heterogenous disorder (8). However, studies of aqueous or vitreous humor acquired from glaucoma patients during surgical intervention point to some key molecular pathways that may play central roles in the pathophysiology of this disease irrespective of the genetic heterogeneity. Specifically, aqueous or vitreous humor from glaucoma patients has been found to contain elevated levels of transforming growth factor beta 2 (TGFβ2) (9-17). TGFβ2 is known to play an important role in the metabolism of extracellular matrix (ECM) and the epithelial to mesenchymal transition (EMT) of fibroblasts (18, 19). Thus, elevated TGFβ2 may play an important role in ECM remodeling and fibrosis at the trabecular meshwork. EMT is known to result in an increase in alpha smooth muscle actin (α-SMA) and, coincidentally, increased expression of α-SMA has been documented in the aqueous humor of glaucoma patients (14). In children, early lensectomy is believed to result in a tsunami of lens epithelial cells onto the anterior surface of the iris and the trabecular meshwork. Previously, it has been shown that TGFβ2 is a key player in EMT of lens epithelial cells (20) and we have proposed a hypothesis for the role of TGFβ2 in the pathogenesis of IAG (21).

A key negative regulator of TGFβ2 is the small leucine rich proteoglycan decorin (22). Aqueous humor from glaucoma patients has been found to have reduced levels of decorin relative to that of normal individuals (14, 23). Furthermore, anti-glaucoma medications have been found to lower decorin and alter profibrotic proteins including TGFβ2 in human tenon's fibroblasts (24). These and other observations collectively suggest that the TGFβ2-decorin axis may play a significant role in the pathophysiology and treatment of glaucoma.

The TGFβ2-decorin axis has recently been studied in animal models of glaucoma. Mice in which the decorin gene has been deleted exhibit a significant increase in IOP (23). Loss of decorin also resulted in loss of optic nerve axons and morphological changes in the glial lamina: typical features observed in glaucoma patients (23). Furthermore, injection of TGFβ2 into the eyes of rats has been found to cause an increase in IOP that could be subsequently inhibited by injections of decorin (25). In addition, adenovirus mediated gene transfer of TGFβ2 in mice or rats has been found to result in an increase in IOP and lead to reduced outflow of aqueous humor through the trabecular meshwork (26). Open-angle IAG is associated with lensectomy and is believed to be caused by the release of lens epithelial cells during cataract surgery that undergo TGFβ2-driven EMT at the trabecular meshwork (20). Thus, while POAG and IAG likely have a different origin of EMT and fibrosis at the trabecular meshwork, the TGFβ2-decorin axis appears to play a central role in both these related indications.

Glaucoma is a chronic disease that needs treatment for long periods of time. The ocular surface poses significant barriers to drugs targeting the trabecular meshwork, and 80 to 90% of drug applied topically to the eye drains through the lacrimal ducts (27). Some drugs that are effective in glaucoma may have significant systemic consequences (28). An alternative approach for the treatment of glaucoma is gene therapy, whereby continuous local production of a therapeutic may overcome the above limitations of topically applied drugs to the eye. The majority of preclinical gene therapy studies in the field of glaucoma have targeted preservation of RGCs through the use of signaling neuroprotective molecules (29). However, such approaches do not address the primary source of pathophysiology in this disease. Accordingly, there remains a need in the art for improved methods for treating glaucoma.

### SUMMARY

In a first aspect, the present disclosure provides methods of treating an eye disease in a subject. The methods comprise administering a recombinant adeno-associated virus (AAV) vector to an eye of the subject. The AAV vector may include (a) a viral capsid protein into which an IKV peptide comprising SEQ ID NO: 1 has been inserted, and (b) a polynucleotide comprising a promoter operably linked to a sequence encoding a decorin protein.

In a second aspect, the present disclosure provides animal models of glaucoma and methods of making an animal model of glaucoma. The animal models of glaucoma are generated by administering a recombinant AAV vector to an eye of an animal. The AAV vector may include (a) a viral capsid protein into which an IKV peptide comprising SEQ ID NO: 1 has been inserted, and (b) a polynucleotide comprising a promoter operably linked to a sequence encoding a constitutively active variant of transforming growth factor beta 2 (TGFβ2^{CS}).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D demonstrate that the vector AAV-IKV mediates gene delivery to the cornea and trabecular meshwork. FIG. 1A: Whole eye scan of a six-week-old C57BL/6J mouse injected intracamerally with AAV-IKV-GFP showing GFP expression in the endothelium, stroma, and trabecular meshwork. FIG. 1B: Higher power imaging reveals that GFP expression is primarily in the corneal stroma, ciliary body, and trabecular meshwork, and that there is also some limited GFP expression is in the corneal endothelium. FIG. 1C: GFP is also present in some of the corneal nerves. FIG. 1D: Bright field images merged with GFP signal reveal punctate GFP in the pigmented ciliary process and iris. TM, trabecular meshwork; CB, ciliary body; I, iris; BF, bright field; GFP, green fluorescent protein channel.

FIGS. 2A-2B demonstrate that transforming growth factor beta 2 (TGFβ2) mediates an increase in intraocular pressure (IOP). FIG. 2A: TGFβ2 expression was analyzed in frozen anterior chamber sections by immunohistochemistry approximately one week after infection with an AAV-IKV vector that expresses a constitutively active variant of TGFβ2 (AAV-IKV-TGFβ2^{CS}). Relative to uninjected animals, AAV-IKV-TGFβ2^{CS} injected animals express significantly greater levels of TGFβ2 in the ciliary body and trabecular meshwork. Some TGFβ2 is elevated in AAV-IKV-pA injected animals due to either the AAV backbone or the injection procedure itself. FIG. 2B: IOP increases within one week after injection of AAV-IKV-TGFβ2^{CS}. Intracameral injection of AAV-IKV-pA did not result in a statistically significant change in IOP relative to uninjected animals at any time point (see FIGS. 4A-4B).

FIGS. 3A-3D demonstrate that TGFβ2^{CS} induces fibrosis at the trabecular meshwork. FIG. 3A: Frozen sections from AAV-IKV-TGFβ2^{CS} injected animals stained for α-smooth muscle actin (α-SMA) and fibronectin 44 days post injection. FIG. 3B: Staining for α-SMA and fibronectin was undetectable under the same conditions in uninjected animals. FIGS. 3C-3D: Frozen sections from AAV-IKV-pA injected animals stained for α-smooth muscle actin (α-SMA) (FIG. 3C) and fibronectin (FIG. 3D) 44 days post injection.

FIGS. 4A-4B demonstrate that AAV-IKV-mediated delivery of decorin inhibits AAV-IKV-TGFβ2^{CS} induced IOP. FIG. 4A: AAV-IKV-Decorin or AAV-IKV-pA were injected intracamerally into six-week-old C57BL/6J mice and stained for decorin after 7 days post injection. While there is a detectable level of decorin in AAV-IKV-pA injected eyes in the ciliary body, there is a significantly higher level of decorin expression in the AAV-IKV-Decorin injected eyes. FIG. 4B: While there was no statistically significant difference in IOP between the AAV-IKV-TGFβ2^{CS} and AAV-IKV-TGFβ2^{CS}+AAV-IKV-Decorin groups initially, i.e. at day 8 (p=0.13) and day 16 (p=0.7), there was a sustained lowering in IOP in the AAV-IKV-TGFβ2^{CS}+AAV-IKV-Decorin injected group relative to the AAV-IKV-TGFβ2^{CS} injected group at all subsequent time points examined (p<0.0001, D23; p<0.0001, D30; p=0.016, D37; p=0.005, D44).

FIGS. 5A-5B demonstrate that decorin inhibits TGFβ2^{CS}-induced fibrosis at the trabecular meshwork. FIG. 5A: Frozen sections from AAV-IKV-TGFβ2^{CS} injected animals stained for α-SMA and fibronectin 44 days post injection. FIG. 5B: Frozen sections from AAV-IKV-TGFβ2^{CS}+AAV-IKV-Decorin injected animals stained for α-SMA and fibronectin. Staining for α-SMA and fibronectin was significantly less in AAV-IKV-TGFβ2+AAV-IKV-Decorin injected groups.

FIGS. 6A-6B demonstrate that decorin inhibits retinal ganglion cell death. FIG. 6A: Frozen retinal sections stained for BRN3a for quantitation of retinal ganglion cells (RGCs) in AAV-IKV-TGFβ2^{CS} and AAV-IKV-TGFβ2^{CS}+AAV-IKV-Decorin injected groups as well as uninjected or AAV-IKV-pA injected animals. There was no statistically significant difference in BRN3a staining between uninjected and AAV-IKV-pA injected animals. FIG. 6B: There was a statistically significant greater number of RGCs in the AAV-IKV-TGFβ2^{CS}+AAV-IKV-Decorin injected group relative to the AAV-IKV-TGFβ2^{CS} injected group (p=0.0322).

FIGS. 7A-7H demonstrate AAV-IKV-mediated gene delivery to the non-human primate cornea. FIG. 7A: GFP expression in the eyes of live African green monkey (Chlorocebus sabaeus) at baseline, day 17, and day 37 post intracameral injection of AAV-IKV-GFP+Nuc1 in the oculus dexter (OD, right eye) and oculus sinister (OS, left eye). While there was no detectable GFP fluorescence prior to injection (baseline), at day 14 post injection, there was clear evidence of GFP expression in the cornea in a punctate pattern of expression (e.g., arrowheads) in live animals. Images containing arrowheads are a higher magnification of the boxed areas shown in the images to their left. At all timepoints, the cornea of both eyes exhibited transparency and lack of any significant signs of inflammation, suggesting that the research grade vector did not lead to any significant immune responses. FIG. 7B: Frozen cryosections examined for expression of GFP at day 45 post injection revealed GFP-positive cells in the cornea. FIG. 7C: IOP in AAV-IKV-GFP+Nuc1 injected eyes relative to baseline at day 14 and day 37 post injection. FIG. 7D: High resolution optical coherence tomography (OCT) imaging of cornea, sclera, and anterior chamber angles of AAV-IKV-GFP injected eyes at day 14 (middle) and day 37 (bottom) were similar to baseline (top). FIGS. 7E-7G: Volume scans revealed that the average thickness of the retina at day 14 (FIG. 7F) and day 37 (FIG. 7G) was similar to baseline (FIG. 7E). FIG. 7H: High resolution OCT imaging of posterior segment retina of AAV-IKV-GFP injected eyes at day 14 (middle) and day 37 (bottom) was also similar to baseline (top).

FIG. 8 demonstrates AAV-IKV mediated gene delivery to the non-human primate cornea. Anterior color, fundus color, and fluorescence images of AAV-IKV-GFP+Nuc1 injected non-human primate eyes at day 14, 37, and 40 post injection. There is no evidence of GFP expression in the fundus at day 14 and day 37 post injection. However, there is a minor GFP signal at day 40 post injection in both the oculus dexter (OD, right eye) and oculus sinister (OS, left eye).

FIGS. 9A-9B show expression of TGFβ2 in frozen ocular section. FIG. 9A: Normal section and needle damage section taken from the eyes of mice where the lens was punctured by a 33-gauge needle. FIG. 9B: In some mice, the lens epithelial cells accumulate as clumps on the surface of the lens.

### DETAILED DESCRIPTION

The present disclosure provides methods of treating an eye disease by administering a recombinant adeno-associated virus (AAV) vector that encodes the proteoglycan decorin to the eye of a subject. Also provided are animal models of glaucoma that are generated using a recombinant AAV vector that encodes a constitutively active variant of transforming growth factor beta 2 (TGFβ2^{CS}).

One challenge in generating a gene therapy for delivery to the trabecular meshwork is the limited availability of potent AAV vectors that target the trabecular meshwork. Recently, the present inventor described a novel AAV vector, referred to as “AAV-IKV,” that delivers transgenes to the outer retina following intravitreal injection (30) and demonstrated that infection of retinal cells by AAV-IKV could be significantly enhanced through the use of a chaperone protein referred to as “Nuc1” (30, 31). See International Patent Application No. WO2021034418, which is hereby incorporated by reference in its entirety. In the present disclosure, the inventor demonstrates that AAV-IKV vector can also infect cells of the anterior chamber (including the ciliary body, trabecular meshwork, and cornea) following intracameral injection. Further, he demonstrates that AAV-IKV mediated gene transfer of human TGFβ2 may be utilized to model pathophysiological aspects of glaucoma, and he investigates whether such a model may be utilized to evaluate AAV-IKV driven expression of decorin as a potential gene therapy for glaucoma. Finally, he demonstrates that AAV-IKV coupled with Nuc1 is tolerated in non-human primate (NHP) eyes as a first step towards clinical development of a gene therapy for glaucoma.

### Methods of Treating Eye Disease:

In a first aspect, the present disclosure provides methods of treating an eye disease in a subject. The methods comprise administering a recombinant AAV vector to an eye of the subject. The AAV vector comprises (a) a viral capsid protein into which an IKV peptide comprising SEQ ID NO: 1 has been inserted, and (b) a polynucleotide comprising a promoter operably linked to a sequence encoding a decorin protein.

The disclosed methods offer several advantages over known methods for treating glaucoma. In the present disclosed methods, the therapeutic protein decorin is delivered in the form of a gene therapy, and a gene encoding decorin is integrated into the genome of cells infected by a recombinant AAV vector. Thus, these methods allow decorin to be continuously produced by infected cells, resulting in greater longevity of expression as compared to methods in which decorin is delivered as a plasmid or protein and avoiding the need for repeated injections. Further, these methods can be used to deliver decorin to eye tissues that would not be accessible via topical administration because the cornea is impermeable to large molecules such as decorin.

Adeno-associated virus (AAV) is a member of the family of Parvoviridae and the genus Dependoparvovirus, and it consists of a single-stranded 4.7 kb DNA genome packed in a non-enveloped capsid of 60 proteins arranged in a T=1 icosahedral symmetry. AAV has been consistently described as a relatively benign viral vector that does not cause significant inflammation in the eye relative to other viruses.

To date, twelve serotypes of AAV have been identified. These serotypes are referred to as AAV1, AAV2, AAV3, etc. The serotypes differ in their tropism (i.e., types of cells they infect), making AAV a very useful system for preferentially transducing specific cell types. Researchers have further refined the tropism of AAV through pseudotyping, i.e., mixing of a capsid and genome from different viral serotypes. The serotypes of pseudotyped viruses are denoted using a slash. For example, AAV2/5 refers to a virus containing the genome of serotype 2 packaged in the capsid from serotype 5. Use of these pseudotyped viruses can also improve transduction efficiency, as well as alter tropism. For example, AAV2/5 targets neurons that are not efficiently transduced by AAV2/2 and is distributed more widely in the brain. In the Examples, the inventor utilized an AAV2 vector pseudotyped with an AAV9 capsid (AAV2/9). However, any AAV serotype, including but not limited to AAV2/2, AAV2/8, or AAV2/5, may be utilized with the present invention (Biotechnol Bioeng 113 (12): 2712-2724, 2016).

As used herein, a “viral vector” is a recombinant (i.e., artificially constructed) viral nucleic acid that has been engineered to encode at least one polynucleotide or polypeptide from a heterologous source.

The AAV vectors of the present disclosure are referred to as “AAV-IKV” because they comprise a viral capsid protein into which an IKV peptide has been inserted. An “IKV peptide” is a shorter portion of the Nuc1 peptide (discussed below) that comprises only the targeting peptide derived from laminin-1, i.e., ASIKVAVSA (SEQ ID NO: 1). In the AAV vectors tested in the Examples, this sequence (SEQ ID NO: 1) is flanked by two glycine residues to form the sequence GASIKVAVSAG (SEQ ID NO: 6). Thus, in some embodiments, the IKV peptide comprises SEQ ID NO: 6 or comprises a peptide that has at least 85%, at least 90%, at least 95%, at least 98%, at least 99%, or at least 99.9% sequence identity to SEQ ID NO: 6. However, the terminal glycine residues are included in SEQ ID NO: 6 to serve as flexible linkers, and they may be altered to comprise different amino acid residues or to be more than one amino acid in length. In previous work, the inventor demonstrated that inclusion of the IKV peptide in the capsid of the AAV vector increases the rate of viral delivery into target cells (see WO2021034418). This increased potency is advantageous because it reduces the need for high doses of virus. Accordingly, the recombinant AAV vectors used with the present invention comprise an IKV peptide.

The IKV peptide may be inserted into any viral capsid protein within the AAV vector. A “viral capsid protein” is a component of the viral protein shell (i.e., capsid). In the Examples, the inventor inserted the IKV peptide into the AAV9 capsid protein VP1 (SEQ ID NO: 11). Thus, in some embodiments, the viral capsid protein is VP1. Specifically, the inventor inserted the IKV peptide of SEQ ID NO: 6 between amino acids 588 and 589 of VP1, forming the construct of SEQ ID NO: 12. However, other suitable sites for insertion of heterologous sequences into AAV capsid proteins have been previously described and may be utilized.

AAV vector delivery is an efficacious method of delivering gene therapies to human cells. In gene therapy, a polynucleotide encoding a gene product is included in an expression construct that further includes a promoter (i.e., to direct the expression of the gene product within the target cells). When the gene product is expressed in a target cell it supplies a desired function. Accordingly, the disclosed AAV vectors are designed to deliver polynucleotides comprising a promoter operably linked to a sequence encoding a protein (i.e., decorin or TGFβ2^{CS}) to target cells.

Any method of protein detection may be used to test whether a cell expresses a protein disclosed herein. Suitable methods for detecting proteins include, without limitation, immunostaining, enzyme-linked immunoassay (ELISA), dot blotting, western blotting, flow cytometry, mass spectrometry, and chromatographic methods.

The terms “polynucleotide,” “oligonucleotide,” and “nucleic acid” are used interchangeably to refer to a polymer of DNA or RNA. A polynucleotide may be single-stranded or double-stranded and may represent the sense or the antisense strand. A polynucleotide may be synthesized or obtained from a natural source. A polynucleotide may contain natural, non-natural, or altered nucleotides, as well as natural, non-natural, or altered internucleotide linkages (e.g., phosphoroamidate linkages, phosphorothioate linkages). The term polynucleotide encompasses constructs, plasmids, vectors, and the like.

As used herein, the term “promoter” refers to a DNA sequence that regulates the expression of a gene. Typically, a promoter is a regulatory region that is capable of binding RNA polymerase and initiating transcription of a downstream (3′ direction) sequence. However, a promoter may be located at the 5′ or 3′ end, within a coding region, or within an intron of a gene that it regulates. Promoters may be derived in their entirety from a native gene, may be composed of elements derived from multiple regulatory sequences found in nature, or may comprise synthetic DNA segments. It is understood by those skilled in the art that different promoters may direct the expression of a gene in different tissues or cell types, at different stages of development, or in response to different environmental conditions. Suitable promoters for use with the present invention include, but are not limited to, constitutive, inducible, temporally regulated, developmentally regulated, chemically regulated, tissue-preferred, and tissue-specific promoters. The promoter may be a plant, animal, bacterial, fungal, or synthetic promoter. A promoter is “operably linked” to a polynucleotide if the promoter is connected to the polynucleotide such that it may affect transcription of the polynucleotide. In the Examples, the inventor utilized the constitutive chicken β-actin promoter in their vectors (i.e., to drive expression of GFP, TGFβ2^{CS}, and decorin). Thus, in some embodiments, the promoter is a constitutive promoter. In some embodiments, the promoter is a chicken β-actin promoter. In some embodiments, the promoter drives expression in a specific cell type found in the eye, for example the cornea.

For use in the methods of treating glaucoma described herein, the AAV vectors are designed to deliver a polynucleotide encoding the therapeutic protein decorin. Decorin is a small leucine-rich proteoglycan (i.e., heavily glycosylated protein) that is a key negative regulator of transforming growth factor b2 (TGFβ2; discussed below). In the Examples, the inventor demonstrate that expression of decorin from an AAV-IKV vector (referred to as AAV-IKV-Decorin) attenuates pathophysiological features of glaucoma in a mouse model of glaucoma. In the Examples, the inventor used the human decorin protein (SEQ ID NO: 9). Thus, in some embodiments, the decorin protein comprises or consists of SEQ ID NO: 9. In some embodiments, the decorin protein has at least 85%, at least 90%, at least 95%, at least 98%, at least 99%, or at least 99.9% sequence identity to SEQ ID NO: 9. However, any decorin protein may be utilized in the AAV-IKV vectors of the present invention. One example of a DNA sequence encoding SEQ ID NO: 9 is provided as SEQ ID NO: 10.

“Percentage of sequence identity” is determined by comparing two optimally aligned sequences over a comparison window. The aligned sequences may comprise insertions or deletions (i.e., gaps) relative to each other for optimal alignment. The percentage is calculated by determining the number of matched positions at which an identical nucleic acid base or amino acid residue occurs in both sequences, dividing the number of matched positions by the total number of positions in the window of comparison and multiplying the result by 100. Protein and nucleic acid sequence identities can be evaluated using the Basic Local Alignment Search Tool (“BLAST”), which is well known in the art (Karlin and Altschul, Methods for assessing the statistical significance of molecular sequence features by using general scoring schemes. Proc. Natl. Acad. Sci. USA (1990) 87:2267-2268; Altschul et al. Gapped BLAST and PSI-BLAST: a new generation of protein database search programs. Nuc1. Acids Res. (1997) 25:3389-3402). The BLAST programs identify homologous sequences by identifying similar segments between a query amino acid or nucleic acid sequence and a test sequence, which is preferably obtained from a protein or nucleic acid sequence database. The BLAST programs can be used with the default parameters or with modified parameters provided by the user.

In the Examples, the recombinant, cell-penetrating peptide Nuc1 is used to increase the delivery of the AAV vector to cells, such as in the anterior chamber of the eye. In previous work, the inventor demonstrated that Nuc1 efficiently penetrates the retina and can deliver a diverse array of agents to ocular tissues (see WO2021034418). Thus, in some embodiments, the methods further comprise administering a Nuc1 peptide to the subject.

For most drugs, the plasma membrane represents an impermeable barrier. “Cell-penetrating peptides” (CPPs) are a special class of proteins that can cross the intact plasma membrane and can facilitate the uptake of cargo molecules. Thus, CPPs enable the delivery of biologically active agents (e.g., proteins, peptides, DNAs, siRNAs, and small molecule drugs) into cells. CPPs offer case of preparation, rapid uptake, and low toxicity and immune response. CPPs generally require chemical conjugation to a cargo molecule for delivery across the plasma membrane, and such modifications can negatively impact the function of the cargo molecule. However, Nuc1 is exceptional in that it does not need to be chemically conjugated or linked to an agent to effectively deliver the agent into a cell (see WO2021034418).

Nuc1 peptides comprise two amino acid sequences selected for their potential ability to target ocular cells. The first of these sequences, ASIKVAVSA (SEQ ID NO: 1), is derived from a longer sequence, CSRARKQAASIKVAVSADR (SEQ ID NO: 4), which is the nucleolin binding region of the human basement membrane glycoprotein laminin-1. The second sequence, DKPRR (SEQ ID NO: 2), is derived from a slightly longer sequence, CDKPRR (SEQ ID NO: 5), which is the heparan sulphate binding domain of a particular isoform of human vascular endothelial growth factor (VEGF165). Importantly, both nucleolin and heparan sulphate are found on the surface of retinal tissues, which likely accounts for the ability of Nuc1 to access these tissues.

In the Nuc1 peptides of the present disclosure, the two ocular-targeting peptides (i.e., SEQ ID NO: 1 and SEQ ID NO: 2) are separated by a flexible linker. A “linker” is a sequence of amino acid residues that serves to connect peptides via a peptide bond. A “flexible linker” is an amino acid sequence that has no fixed structure (i.e., secondary or tertiary structure) in solution and is, therefore, free to accept a variety of conformations. The linker used with the present disclosure may comprise one or more amino acid residues, preferably 1, 2, 3, 4, 5, 6, 7, 8, 9, 10, 11, 12, 13, 14, 15, 16, 17, 18, 19, or 20 or more residues. In a preferred embodiment of this disclosure, the linker comprises 2, 3, 4, 5, or 6 residues. More preferably, the linker comprises 2 residues. The linker may comprise existing amino acids that are part of one of the ocular-targeting peptides or it may be an unrelated sequence that is inserted between the ocular-targeting peptides. The linker may comprise any amino acid sequence that does not substantially hinder the interaction of the ocular-targeting peptides with their corresponding target molecules. Preferred amino acid residues for flexible linker sequences include glycine, alanine, serine, threonine, lysine, arginine, glutamine, and glutamic acid, but are not limited thereto. The Nuc1 peptide that was tested in the Examples is that of SEQ ID NO: 3, which comprises SEQ ID NO: 1 linked to SEQ ID NO: 2 by a linker comprising two glycine residues. Thus, in some embodiments, the Nuc1 peptide comprises SEQ ID NO: 3 and, in some embodiments, the flexible linker comprises two glycine residues. In some embodiments, the Nuc1 peptide is a peptide that has at least 85%, at least 90%, at least 95%, at least 98%, at least 99%, or at least 99.9% sequence identity to SEQ ID NO: 3. However, those of skill in the art will understand that the flexible linker may comprise other residues and that the length of the flexible linker may be altered without adversely affecting the function of the CPP.

The Nuc1 peptide used in the Examples was synthesized by a commercial supplier of custom peptides. Peptide synthesis may be performed using a solid-phase technique (Science 269:202-204, 1995). However, those skilled in the art will appreciate that other means may be used to generate peptides including, but not limited to, other chemical synthesis methods as well as protein expression systems. The flexible linker may be introduced into the Nuc1 peptide as either a nucleic acid or a peptide. Typically, a nucleic acid encoding the linker is included in the recombinant nucleic acid encoding the Nuc1 peptide prior to protein synthesis. However, the linker can also be prepared separately by peptide synthesis and subsequently combined with the two targeting peptides. Methods of manipulating nucleic acids and methods of peptide synthesis are well known in the art.

The terms “polypeptide,” “protein,” and “peptide” are used interchangeably herein to refer to a series of amino acid residues connected by peptide bonds between the alpha-amino and carboxy groups of adjacent residues, forming a polymer of amino acids. Polypeptides may include modified amino acids (e.g., phosphorylated, glycated, glycosylated, etc.) and amino acid analogs. The terms “protein” and “polypeptide” are often used in reference to relatively large polypeptides, whereas the term “peptide” is often used in reference to small polypeptides, but usage of these terms in the art overlaps.

In the provided methods, the AAV vector, and optionally the Nuc1 peptide, may be administered using any route of administration that is effective for treating the subject. Suitable routes of administration to an ocular tissue include, without limitation, trans-ocular, intravitreal, topical, trans choroidal, intracameral, supra choroidal, transdermal, subretinal, intra-peritoneal, subcutaneous, and intravenous routes. The AAV vector, and optionally the Nuc1 peptide, may also be administered directly into the cornea itself. However, in some preferred embodiments, administration is intracameral. “Intracameral injection” directly delivers the vector into the anterior chamber of the eye. In some embodiments, the Nuc1 peptide and/or AAV vector are administered to only one eye of the subject. In other embodiments, the Nuc1 peptide and/or AAV vector are administered to both eyes of the subject.

The exact dosage of the AAV vector and the optional Nuc1 peptide that is administered should be chosen by a physician in view of the subject to be treated. Dosage should be adjusted to provide sufficient levels of decorin expression to maintain the desired effect. Factors that may be considered include the severity of the disease (e.g., extent of the condition, history of the condition); the age, weight, and gender of the patient; time and frequency of administration; diet; drug combinations; reaction sensitivities; and tolerance/response to therapy. For any active agent, the therapeutically effective dose can be estimated initially either in cell culture assays or in animal models, usually mice, rabbits, dogs, or pigs. In some embodiments, between about 0.5×10⁹ and about 5.0×10¹⁰ vector genomes of the AAV vector are administered. In some embodiments, about 0.2-0.7 mg, about 0.3-0.6 mg, or about 0.5 mg of Nuc1 is administered.

For use in the present disclosure, the AAV vector, and optionally the Nuc1 peptide, may be formulated as a pharmaceutical composition that further comprises a pharmaceutically acceptable carrier. Pharmaceutically acceptable carriers include solvents, diluents, or other liquid vehicles, dispersion or suspension aids, surface active agents, isotonic agents, thickening or emulsifying agents, preservatives, solid binders, and lubricants, and combinations of any of these carriers. The pharmaceutical composition may also comprise one or more additional therapeutic agents such as growth factors, anti-inflammatory agents, vasopressor agents, collagenase inhibitors, topical steroids, matrix metalloproteinase inhibitors, ascorbates, angiotensin II, angiotensin calreticulin, tetracyclines, fibronectin, collagen, thrombospondin, B vitamins, and hyaluronic acid.

As used herein, “treating” describes something that is done to a subject to combat a health problem (i.e., a disease, condition, or disorder). Treating may involve controlling the health problem, lessening its symptoms or complications, slowing its progression, and/or eliminating it.

The disclosed methods are used to treat an eye disease. Common eye diseases include age-related macular degeneration, retinitis pigmentosa, and glaucoma, which are associated with degeneration of the retinal pigment epithelium, photoreceptors and retinal ganglion cells, respectively (Lancet 368:1795-1809, 2006; Nat Rev Neurosci 7:860-872, 2006). Other eye diseases and conditions include retinal tear, retinal detachment, diabetic retinopathy, epiretinal membrane, macular hole, macular degeneration, bulging eyes, cataracts, CMV retinitis, retinoblastoma, diabetic macular edema, ocular hypertension, ocular migraine, retinal detachment, alkali or other chemical burn, hyphema, corneal abrasion, keratitis, keratoconus, subconjunctival hemorrhage, proliferative vitreoretinopathy, Usher syndrome, and uveitis. Any eye disease that involves fibrosis or elevated TGFβ2 may be treated using the methods of the present invention. Examples of fibrotic disorders of the eye include, but are not limited to, scarring in the cornea and conjunctiva, fibrosis of the lens capsule post-cataract surgery, and proliferative vitreoretinopathy. In preferred embodiments, the eye disease is glaucoma. “Glaucoma” is a group of eye diseases in which abnormally high intraocular pressure damages the optic nerve. In specific embodiments, the glaucoma is primary open angle glaucoma (POAG) or infantile aphakic glaucoma (IAG). “Primary open-angle glaucoma” (POAG) is a type of glaucoma that is characterized by an open, normal appearing anterior chamber angle and raised intraocular pressure (IOP). “Infantile aphakic glaucoma” (IAG) is a type of glaucoma that develops in infants after they have undergone cataract surgery.

The “subject” to which the methods are applied may be any vertebrate. Suitably, the subject is a mammal, such as a human, cow, horse, sheep, pig, goat, rabbit, dog, cat, bat, mouse, or rat. In certain embodiments, the methods may be performed on lab animals (e.g., mice and rats) for research purposes. In other embodiments, the methods are used to treat commercially important farm animals (e.g., cows, horses, pigs, rabbits, goats, sheep, and chickens) or companion animals (e.g., cats and dogs). In a preferred embodiment, the subject is a human.

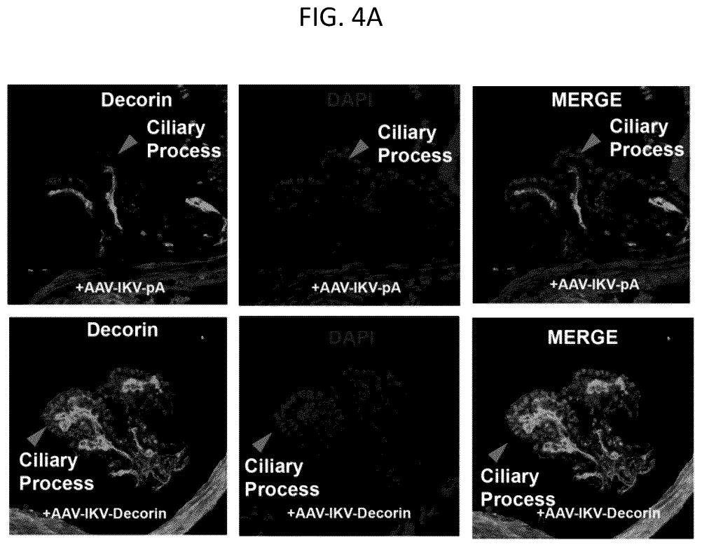

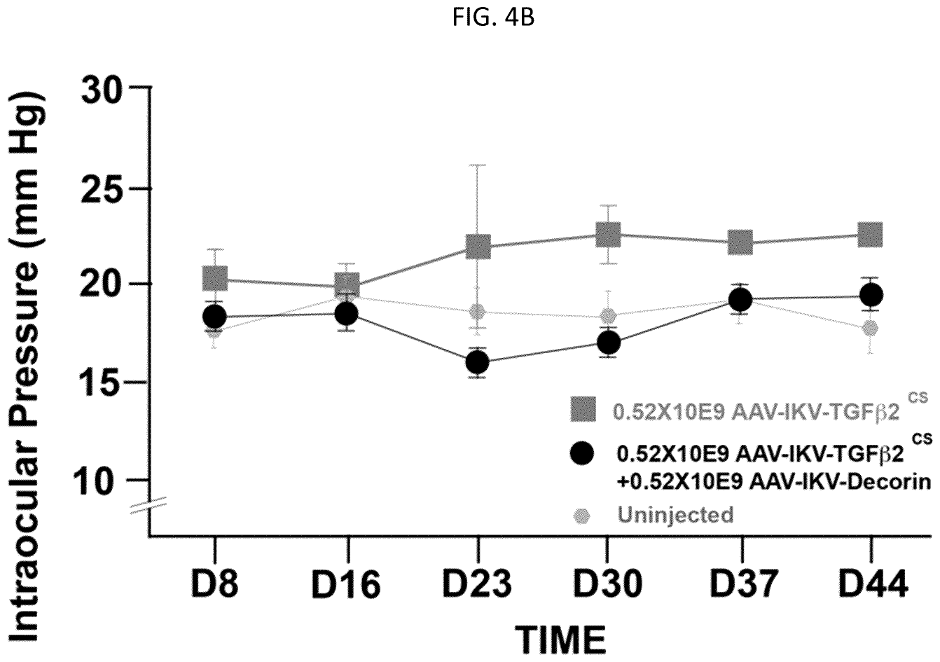

The disclosed methods are designed to deliver the therapeutic protein decorin to the anterior chamber of the eye to treat eye diseases such as glaucoma. The “anterior chamber” is an aqueous humor-filled space in the eye that is located between the cornea and the iris. The inventor has determined that decorin is expressed in the trabecular meshwork, ciliary body, corneal stroma, and corneal endothelium (all within the anterior chamber) at various levels following co-administration of the Nuc1 peptide and the AAV-IKV-Decorin vector (FIG. 4A). Thus, in some embodiments, the decorin protein is expressed in the trabecular meshwork, ciliary body, corneal stroma, and/or corneal endothelium. The “trabecular meshwork” is a spongy tissue in the eye that regulates the flow of aqueous humor. The “ciliary body” is a ring-shaped structure in the eye that produces fluid and controls the shape of the lens. The “corneal stroma” is the thickest layer of the human cornea and is responsible for maintaining the transparency of the cornea and its refractive function. The “corneal endothelium” is a single layer of cells that lines the posterior side of the cornea that transports water from the stroma into the anterior chamber.

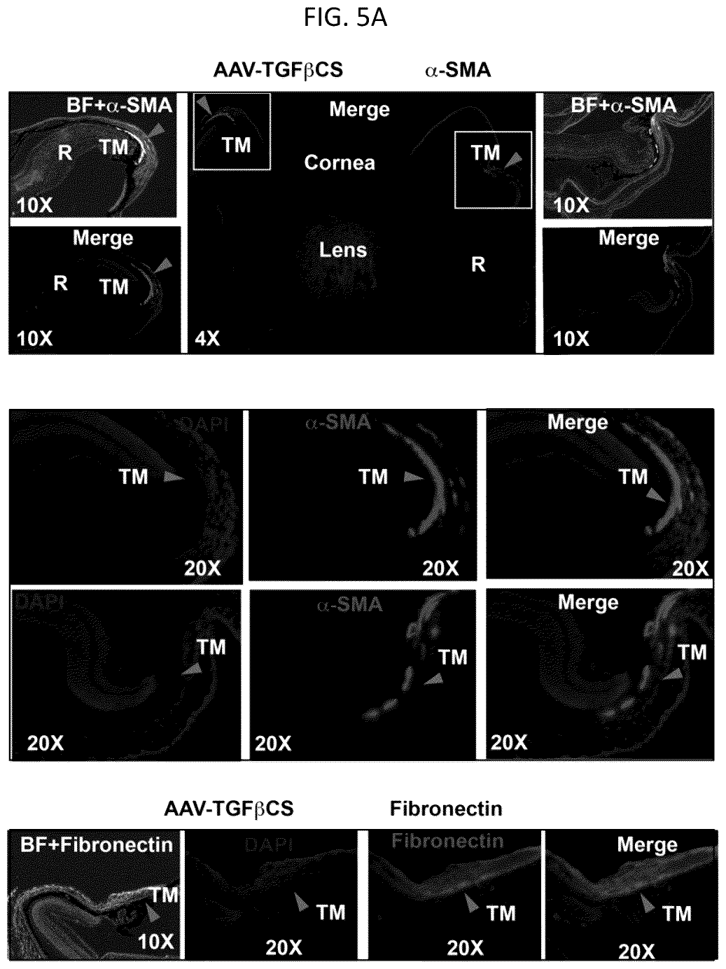

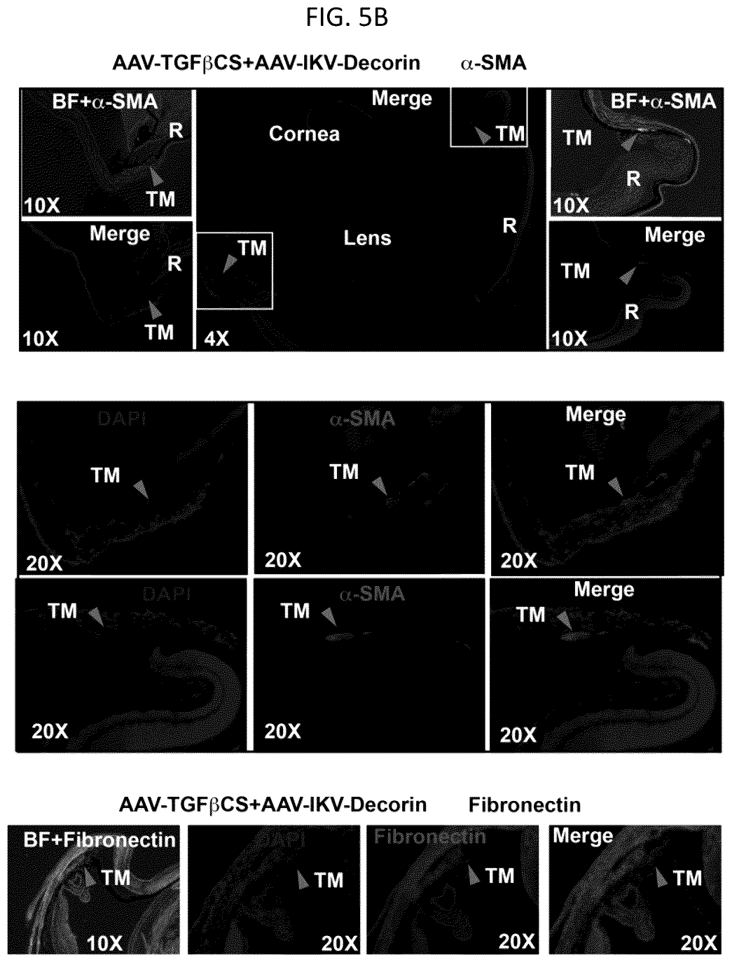

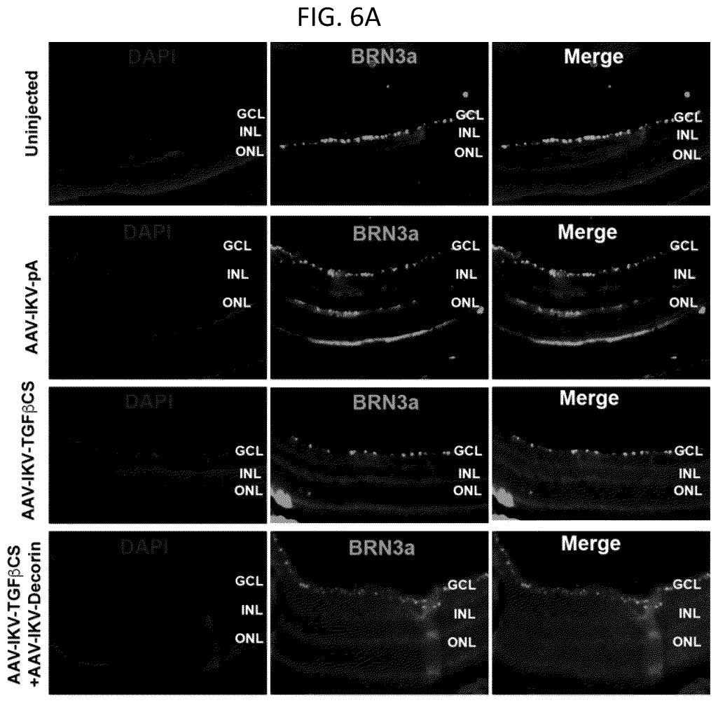

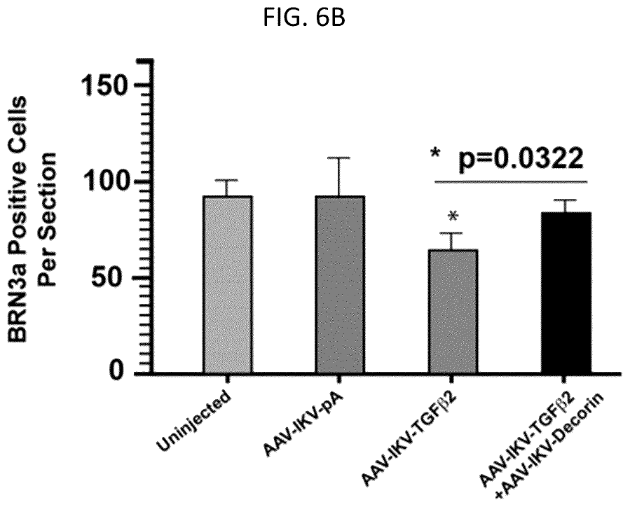

In the Examples, the inventor shows that co-administration of Nuc1 and AAV-IKV-Decorin resulted in (a) sustained reduction of intraocular pressure (IOP) (FIG. 4B); (b) reduced fibrosis at the trabecular meshwork (FIG. 5), and (c) a greater number of retinal ganglion cells (RGCs; FIG. 6) in a mouse model of glaucoma (note: this animal model is described in the following section). Thus, in some embodiments, co-administration of the Nuc1 peptide and AAV vector produces one or more of these outcomes.

“Fibrosis” refers to the development of fibrous connective tissue. In glaucoma, fibrosis of the trabecular meshwork results in attenuation of aqueous humor outflow, which leads to elevated IOP. Fibrosis can be detected via visual assessment of the trabecular meshwork via gonioscopy or can be detected in tissue samples using histological staining techniques, e.g., staining of collagen using Masson's trichrome stain or immunostaining of α-smooth muscle actin (α-SMA). Trabecular meshwork tissue samples can be obtained through a procedure called gonioscopy-assisted transluminal trabeculotomy (GATT).

“Intraocular pressure” (IOP) is a measurement of the fluid pressure inside the eye. IOP can be measured using a tonometer. Increased IOP ultimately leads to retinal ganglion cell (RGC) death. Loss of these cells is the major factor contributing to visual impairment in glaucoma patients. RGC death can be assessed by immunostaining retinal tissue (e.g., staining for the RGC marker BRN3a) and counting the number of surviving RGCs in a specific area. Alternatively, if tissue samples are not obtainable, optical coherence tomography (OCT) can be used to measure retinal nerve fiber layer thickness as a surrogate marker for RGC loss, and visual field testing can be used to evaluate functional deficits associated with RGC damage.

### Animal Model of Glaucoma:

In a second aspect, the present disclosure provides methods of generating an animal model of glaucoma. The animal model is generated by administering a recombinant AAV vector to an eye of an animal. The AAV vector comprises (a) a viral capsid protein into which an IKV peptide comprising SEQ ID NO: 1 has been inserted, and (b) a polynucleotide comprising a promoter operably linked to a sequence encoding a constitutively active variant of transforming growth factor beta 2 (TGFβ2^{CS}).

An “animal model” is a non-human animal that is used in research because it mimics aspects of a disease found in humans. Expression of TGFβ2^{CS} in the animal models of the present disclosure causes them to exhibit pathophysiological features of glaucoma. Specifically, in the Examples, the inventor demonstrated that expression of the TGFβ2^{CS} protein led to fibrosis of the trabecular meshwork in mice and to a subsequent increase in IOP and RGC death. Suitable animals that can be used as an animal model include, without limitation, mice, rats, rabbits, dogs, pigs, and non-human primates. In some embodiments, the animal is a mouse or a non-human primate. In specific embodiments, the non-human primate is an African green monkey (Chlorocebus sabaeus).

The recombinant AAV vectors used to generate animal models of glaucoma differ from the recombinant AAV vectors used in the methods of treating glaucoma (AAV-IKV-Decorin; described in the previous section) only in that they are designed to deliver a polynucleotide encoding a constitutively active form of the protein transforming growth factor beta 2 (TGFβ2^{CS}) rather than a polynucleotide encoding the therapeutic protein decorin. These recombinant AAV vectors used to generate animal models of glaucoma are referred to herein as AAV-IKV-TGFβ2^{CS}.

Transforming growth factor beta 2 (TGFβ2) is a cytokine that is involved in many cellular functions. TGFβ2 affects retinal ganglionic cell survival and is implicated in glaucoma pathogenesis. TGFβ2 is produced as a latent 390-amino acid precursor that forms dimers through association of its N-terminal latency-associated peptide (LAP). The dimeric, mature, biologically active C-terminal 112-amino acid TGFβ2 is generated by the cleavage and dissociation of LAP. A “constitutively active” variant of TGFβ2 (TGFβ2^{CS}) is a variant that is always in an active state, regardless of its cleavage status. In the Examples, the inventor made human TGFβ2 constitutively active by introducing the mutations C226S and C228S into the LAP domain, forming SEQ ID NO: 7. Thus, in some embodiments, the TGFβ2C'S protein comprises SEQ ID NO: 7 or comprises a peptide that has at least 85%, at least 90%, at least 95%, at least 98%, at least 99%, or at least 99.9% sequence identity to SEQ ID NO: 7. One example of a DNA sequence encoding SEQ ID NO: 7 is provided as SEQ ID NO: 8.

As in the methods described above, the recombinant AAV vector can be co-administered with a Nuc1 peptide to increase the efficiency of its delivery. Thus, in some embodiments, a Nuc1 peptide is co-administered with the recombinant AAV vector to an eye of the animal to generate the animal model. In these embodiments, the Nuc1 peptide comprises: (a) SEQ ID NO: 1 linked to SEQ ID NO: 2 via a flexible linker, or (b) SEQ ID NO: 3. In some embodiments, the Nuc1 peptide comprises a peptide that has at least 85%, at least 90%, at least 95%, at least 98%, at least 99%, or at least 99.9% sequence identity to SEQ ID NO: 3.

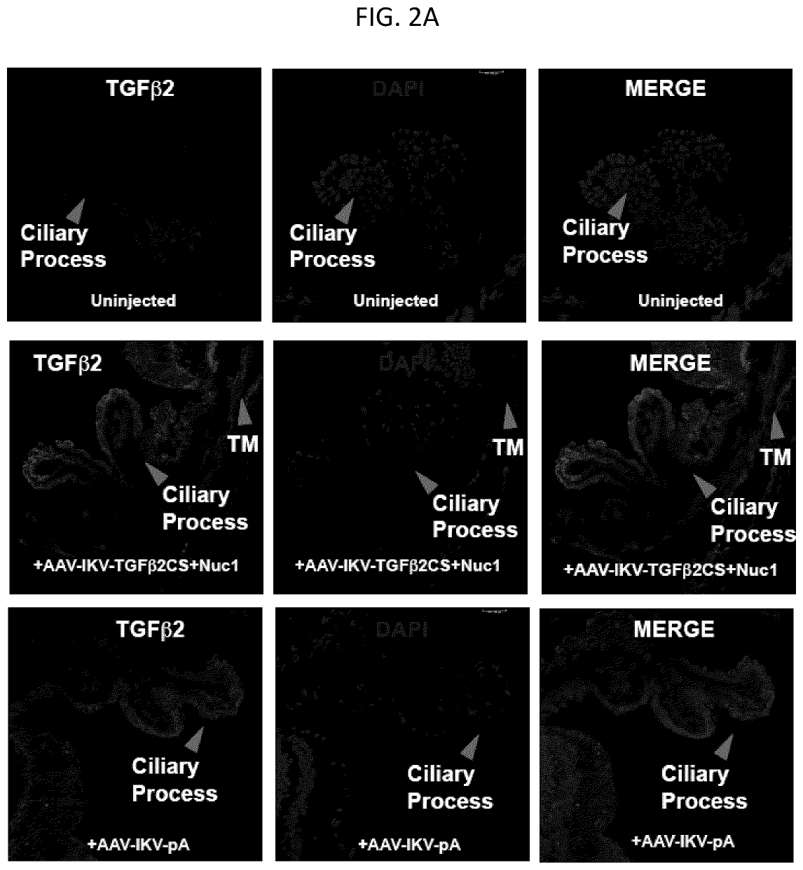

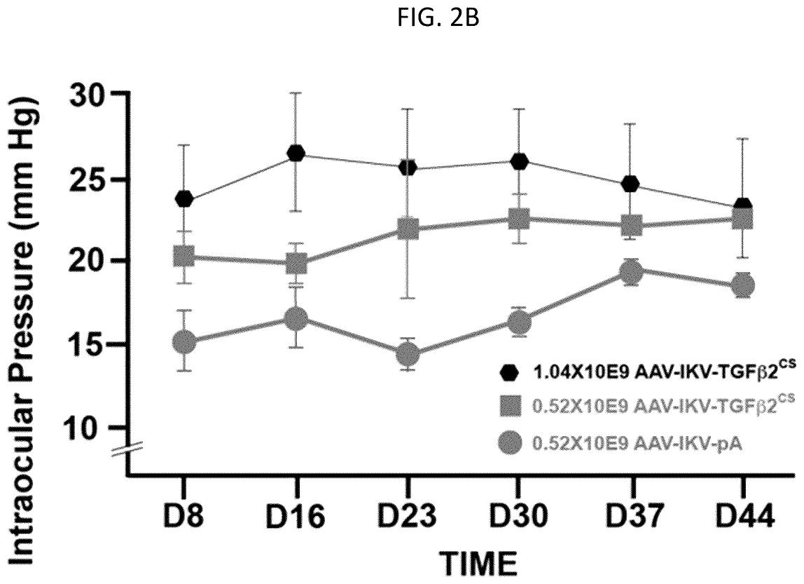

To make an animal model of glaucoma, the AAV-IKV vector encoding TGFβ2C$ was delivered to the anterior chamber of the eye of an animal. In the Examples, the inventor demonstrates that animals express significantly greater levels of TGFβ2 in the ciliary body and trabecular meshwork following co-administration of this AAV-IKV-TGFβ2^{CS} vector and the Nuc1 peptide (FIG. 2A). Thus, in some embodiments, TGFβ2^{CS} is expressed in the ciliary body and trabecular meshwork.

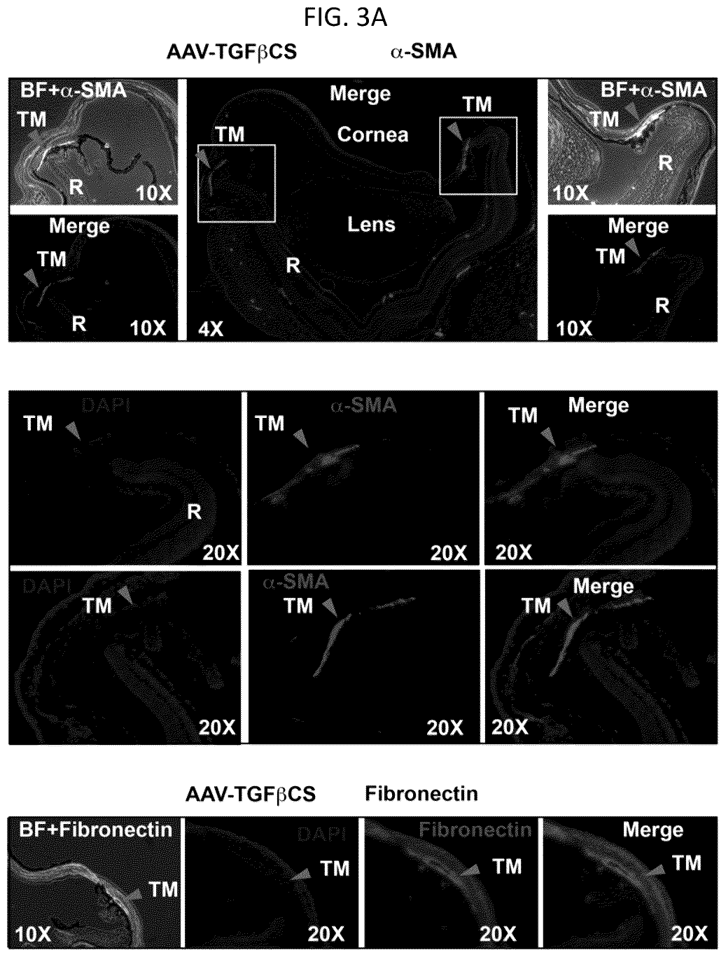

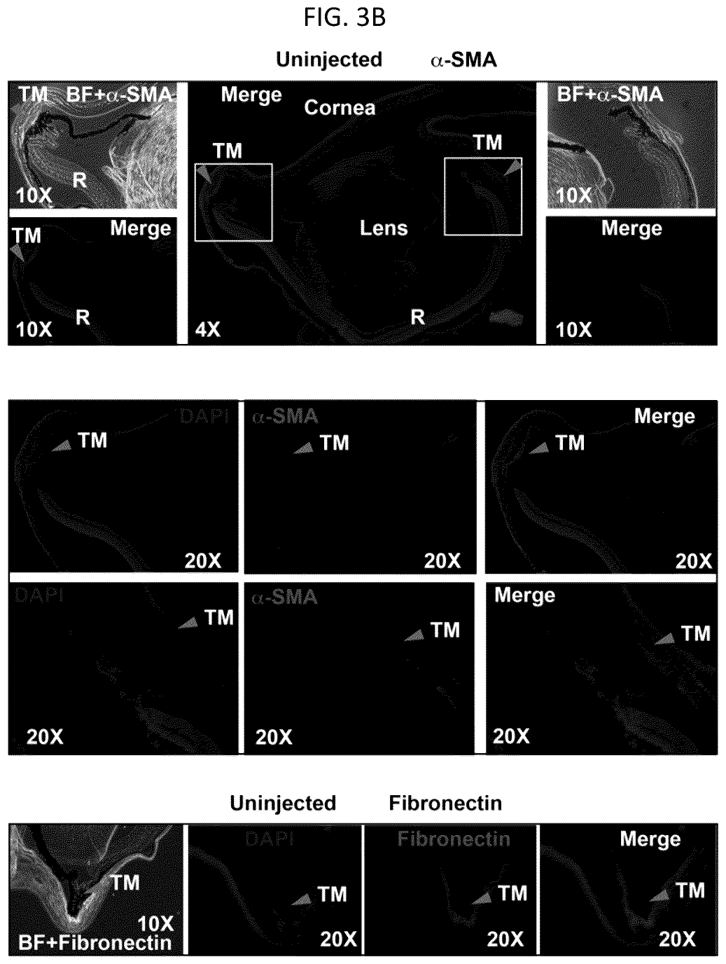

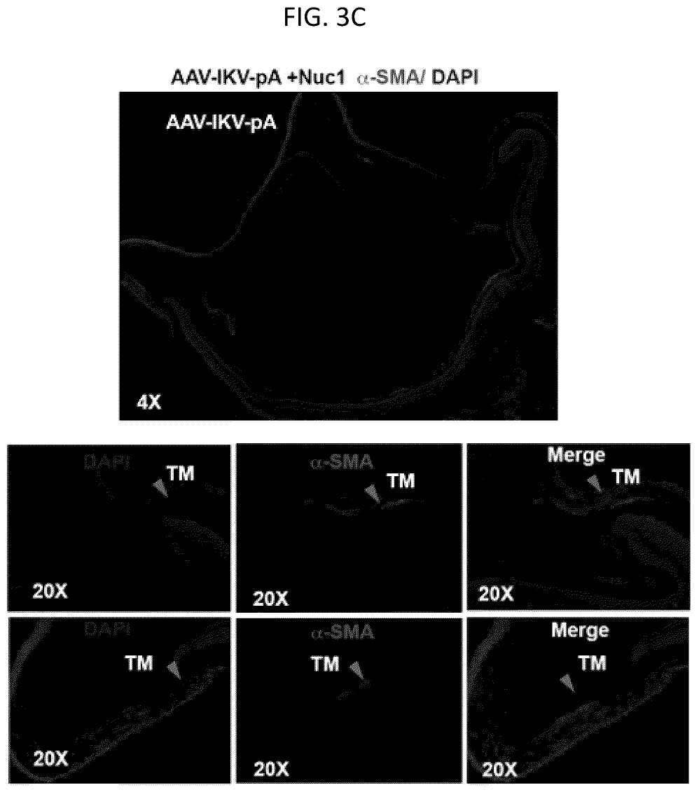

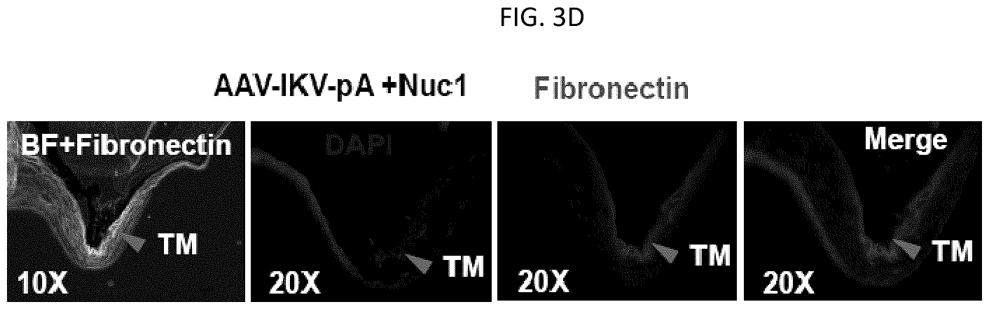

Further, in the Examples, the inventor shows that animals infected with AAV-IKV-TGFβ2^{CS} exhibit (a) increased IOP relative to a control animal (FIG. 2B); (b) increased fibrosis at the trabecular meshwork relative to a control animal (FIG. 3A); and/or (c) increased RGC death relative to a control animal (FIG. 6). Thus, in some embodiments, the animal model exhibits one or more of these characteristics.

As used herein, the term “control animal” refers to a comparable animal (e.g., of the same breed, sex, and age) that was raised under the same or comparable conditions (e.g., diet, weaning protocol) but was not infected with AAV-IKV-TGFβ2^{CS}. In some embodiments, the IOP of the animal model is increased as compared to the TOP of the control animal. In some embodiments, the fibrosis at the trabecular meshwork in the animal model is increased as compared to the fibrosis at the trabecular meshwork in the control animal. In some embodiments, the RGC death in the animal model is increased as compared to the RGC death in the control animal.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the disclosure, invention or of the appended claims.

### Examples

Primary open angle glaucoma (POAG) and infantile aphakic glaucoma (IAG) are two major causes of irreversible vision loss in adults and infants, respectively. Both indications are associated with fibrosis of the trabecular meshwork that results in attenuation of aqueous humor outflow. This reduction in outflow causes an increase in intraocular pressure (IOP) that ultimately leads to retinal ganglion cell (RGC) death. Transforming growth factor b2 (TGFβ2) is implicated in epithelial to mesenchymal transition of cells (EMT) in both POAG and IAG. Decorin is a major negative regulator of TGFβ2, and expression of this proteoglycan is reduced in glaucoma patients.

In the following example, the inventor demonstrates highly efficient infection of the murine anterior chamber including ciliary body, corneal stroma, TM and corneal nerves using a novel AAV vector that is referred to herein as “AAV-IKV”. Intracameral injection of an AAV-IKV vector expressing a constitutively active TGFb2 (AAV-IKV-TGFb2^{CS}) led to fibrosis of the trabecular meshwork in mice and a subsequent increase in IOP and RGC death, modeling pathophysiological features of POAG and IAG. Expression of human decorin from an AAV-IKV vector (AAV-IKV-Decorin) attenuated fibrosis, IOP, and RGC death in AAV-IKV-TGFb2^{CS} injected mice, suggesting that AAV-IKV-Decorin may be a useful therapy for POAG and IAG. Finally, intracameral injection of an AAV-IKV vector expressing GFP in a non-human primate led to GFP expression in the cornea without any discernible immune related toxicities.

### Materials and Methods:

### Animals

Six- to eight-week-old male C57BL/6 mice were purchased from the Jackson Laboratory (Bar Harbor, ME) and housed in an Institutional Animal Care and Use Committee (IACUC) approved animal facility at Tufts University School of Medicine. Animals were housed under a 12-hour light/dark cycle. This study was carried out in accordance with the Statement for the Use of Animals in Ophthalmic and Vision Research, set out by the Association of Research in Vision and Ophthalmology (ARVO). Studies are reported in accordance with ARRIVE guidelines (arriveguidelines.org).

A study in African green monkey (Chlorocebus sabaeus) was conducted in compliance with ARVO Statement for the Use of Animals in Ophthalmic and Vision Research at the St. Kitts Biomedical Research Foundation. Monkeys were drug-naïve and humanely procured from the healthy wild population. Monkeys were observed twice daily in their home cages, assessing for behavioral changes and overall well-being. Additional monitoring was conducted during scheduled ophthalmic examinations. Animals were placed in single-cage housing throughout the study following approval from the IACUC.

Four monkeys were treated with anthelmintics to eliminate intestinal parasite burden and were observed in quarantine for a minimum of 4 weeks prior to screening for study enrollment. All monkeys underwent a minimum of 7 days acclimation to study housing prior to in-life initiation. Prior to study enrollment, a clinical exam was performed on the four monkeys. If healthy by these criteria, monkeys went through ophthalmic screening to identify an optimal animal for study enrollment. Baseline ophthalmic exam included tonometry, slit lamp biomicroscopy, fundoscopy, color fundus photography (CFP), fluorescence fundus photography, and optical coherence tomography (OCT) as described below.

General assessment of the monkeys was conducted a minimum of twice daily and during any sedation or restraint interval to confirm good health and identify abnormalities suggestive of adverse response to treatment.

### Generation of AAV Constructs

The AAV-IKV capsid as well as the Nuc1 chaperone, both utilized in this study have been described previously (30, 31). Methods to construct, amplify and purify AAV-IKV and an AAV-IKV expressing GFP (AAV-IKV-GFP) have also been described previously (30). Specific to this study, an AAV-IKV vector containing a constitutively active TGFβ2 was constructed by mutating cysteine at positions 226 and 228 to serine of the human TGFβ2 propeptide (NP_003229) and replacing the GFP in AAV-IKV-GFP with this mutated TGFβ2. Hereafter, this construct is referred to as AAV-IKV-TGFb2^{CS}. Similarly, a construct expressing human decorin (NM_001920) was also constructed. Similar to GFP, both human transgenes were expressed from a chicken β actin promoter.

### Intracameral Injections

Mice were anesthetized by intraperitoneal injections of a mixture containing 0.1 mg/g body weight ketamine (Phoenix™, St Joseph, MO) and of 0.01 mg/g body weight xylazine (Lloyed, Shenandoah, Iowa) followed by topical application of 0.5% proparacaine hydrochloride (Akorn Inc., Lake Forest, IL, USA) to dilate the pupil. Injections were performed by delivering a total volume of 1.5 μl/eye using a Hamilton glass micro-syringe (Hamilton company, NV, USA) fitted with a custom 33-guage 0.5 inch, point style 4 needle with 30° bevel. After injecting the sample into the anterior chamber, the needle was left in place for 30-60 seconds before the needle was withdrawn. Injections were administered unilaterally, while uninjected eyes served as a contralateral control. Except where indicated, mice were injected with 0.52×10⁹ vector genomes of the respective recombinant AAV virus.

Anesthesia was achieved in NHP with intramuscular ketamine (8 mg/kg) and xylazine (1.6 mg/kg) to effect, and pupil dilation with topical 10% phenylephrine and/or 1% cyclopentolate. For intracameral injection, a speculum was placed in the eye to facilitate injections followed by a drop of proparacaine hydrochloride 0.5% and then 5% Betadine solution, and a rinse with sterile saline. An intracameral (IC) injection containing 1.225×10¹¹ AAV-IKV-GFP vector genomes and 12.5 mg Nuc1 was performed using 31-gauge 0.5-inch-long needle connected to 0.3-mL syringe. The needle was introduced through the temporal cornea ˜2 mm anterior to the limbus without disturbing the intraocular structures. Following completion of the injection, the needle remained inserted for approximately 10 seconds to ensure dispersal of the test article. The animal then received a subconjunctival injection of 50 μl of 40 mg/mL triamcinolone. Following the injection, a topical triple antibiotic neomycin, polymyxin, bacitracin ophthalmic ointment was administered.

### Tissue Processing and Immunostaining

Eyes were enucleated at the end of the study from the respective groups and incubated in a fixative comprising 4% paraformaldehyde (Electron Microscopy Sciences, 15735-50S) at 4° C. overnight. The next day the eyes were washed with PBS and transferred into 15% sucrose solution (in 0.1M sodium phosphate solution, pH 7.4, Boston bioproducts, BB-149) for 5-6 hours at 4° C. and subsequently transferred into 30% sucrose solution for overnight incubation at 4° C. The next day, eyes were processed for snap freezing using O.C.T compound (Tissue Tek, 4583). Cryosections of 14 μm were incubated in either 2% BSA+0.3% Triton X-100 or in 6% normal goat serum+PBS-Triton x-100 for 1 hour at room temperature followed by incubation with a 1:200 dilution of primary antibody at 4° C. overnight. Detection was performed by incubation with a secondary Cy3-conjugated antibody for 1 hour at room temperature. Imaging of stained sections was performed using an Olympus IX51 microscope with appropriate filters. Images were captured using a Retiga 2000r camera. The sources of antibodies and the dilutions used for staining are indicated in Table 1 below. Eyes were fixed in Davidson's fixative solution (Electron Microscopy Sciences, 6413310) for histology. Paraffin embedding, sectioning, and H/E staining was conducted by Tufts Comparative Pathology Services, Boston, MA. Images were acquired by using an Olympus 1×51 microscope.

TABLE 1
Antibodies used for immunohistochemistry

| Antibody | Dilution | Catalog No. | Supplier Name |
|---|---|---|---|
| α-SMA | 1:200 | C6198 | Sigma Aldrich, MO |
| Fibronectin | 1:200 | ab45688 | Abcam, MA |
| Brn3a | 1:50 | sc-8429 | Santacruz, |
| TGFβ2 | 1:200 | MAB73461 | R & D systems, MN |
| Goat anti-rabbit cy3 | 1:500 | 111-165-003 | Jackson |
| | | | ImmunoResearch, PA |
| Goat anti-rat cy3 | 1:500 | A10522 | Invitrogen, CA |

### IOP Measurement in Mice

Baseline intraocular pressure (IOP) was recorded from each mouse eye before administering intracameral injection and was recorded weekly in injected or uninjected eyes following intracameral injection using an iCare Tonolab rebound tonometer (Icare, Finland). The IOP was recorded between 11 AM and 2 PM. This time range was maintained for each of the studies to avoid confounding results due to circadian variability in IOP (32). Mice were anaesthetized before measuring IOP. Six rebound measurements were recorded with the tonometer, measured from the central cornea, to provide an average value of IOP (mm Hg). All IOP measurements were conducted following the manufacturer's recommendations. A standard deviation for six rebound measurements of less than 2.5 (SD<2.5) was considered evidence of accurate recording. The measurements were repeated if the above range was not initially met.

In NHPs, IOP measurements were collected using a TonoVet (iCare, Finland) tonometer set to the dog (d) calibration setting. The Tono Vet rebound tonometer is well tolerated and required no additional local analgesia. Approximately 4 minutes after administration of anesthesia, when full sedation was achieved, monkeys were placed in a supine position in an IOP testing apparatus. Approximately one minute after supine positioning, three measurements were taken from each eye at each time point and the mean was used for IOP analysis.

### Statistical Analysis

Statistical analysis was performed using GraphPad Prism software (San Diego, CA). All p-values were determined by unpaired two-tailed parametric t-test or nonparametric Mann-Whitney U test. One-way or two-way ANOVA followed by Bonferroni's multiple comparison test was used to determine statistically significant differences when comparing the results of more than two or three independent groups. Differences were considered statistically significant as follows: *p≤0.05, **p≤0.01, ***p≤0.001, and ****p≤0.0001. Results are shown as the mean±SEM.

### Ophthalmic Exams and Imaging in Non-Human Primates

One monkey, approximately 9-11 years old and weighing 4.84 kg, was selected for study. Eyes were examined by slit lamp at baseline and at time intervals up to the completion of the study (day 43 post IC injection) to evaluate integrity of the ocular surface and general ocular health, broad ocular response to AAV-IKV-GFP, and normal response to mydriatics (10% phenylephrine HCl and 1% cyclopentolate HCl). Ophthalmic imaging, including color fundus, fluorescence fundus, and OCT imaging, was performed to detect any pathological response to AAV-IKV-GFP.

### Anterior & Fundus Photography in Non-Human Primates

Color anterior and fundus imaging and fluorescent anterior and fundus imaging were performed using a Topcon TRC-50EX retinal camera with Canon 6D digital imaging hardware and New Vision Fundus Image Analysis System software. For the color anterior and fundus photos, the following settings were applied: shutter speed (Tv) 1/25 sec, ISO 400, and Flash 18. For monochromatic anterior and fundus photos the following settings were applied: Tv 1/5 sec, ISO 400, and Flash 25. For fluorescent anterior and fundus images the following settings were applied: exciter and barrier filters engaged (480 nm exciter/525 nm barrier filter), Tv 1/5 sec, ISO 3200, and Flash 300. Fluorescence photographs were qualitatively evaluated to define extent of GFP expression.

### Optical Coherence Tomography in Non-Human Primates

Anterior and posterior segment optical coherence tomography (OCT) were performed oculus uturque (OU, in both eyes) using a Heidelberg Spectralis OCT HRA plus or OCT system with eye tracking and HEYEX image capture and analysis software. An overall volume scan of the entire macula was performed at a dense scan interval. At the time of OCT measurement, the blue laser autofluorescence wide-field function of the Spectralis was used to obtain images of GFP expression in the retina and anterior chamber. When permitted by media clarity, the composite image function was employed to maximize the area of the retina that is captured in each image file.

### Termination and Eye Collection in Non-Human Primates

Animals were terminated on Day 45 after review of Day 43 images via euthanization with sodium pentobarbital (25-30 mg/kg IV). Globes were enucleated after marking the 12:00 o'clock position with a limbal suture. Thereafter, an approximately 8 mm limbal incision was made, followed by trimming of excess orbital tissue with retention of the optic nerve. Globes were transferred to 4% paraformaldehyde solution (prepared in PBS) for 4 hours at 4° C. and were then thoroughly washed in PBS three times for 15 minutes each. Globes were then immersed in 30% sucrose solution (prepared in PBS) at 4° C. and shipped for further analysis.

### Results:

### AAV-IKV Mediated Gene Delivery to the Murine Anterior Segment

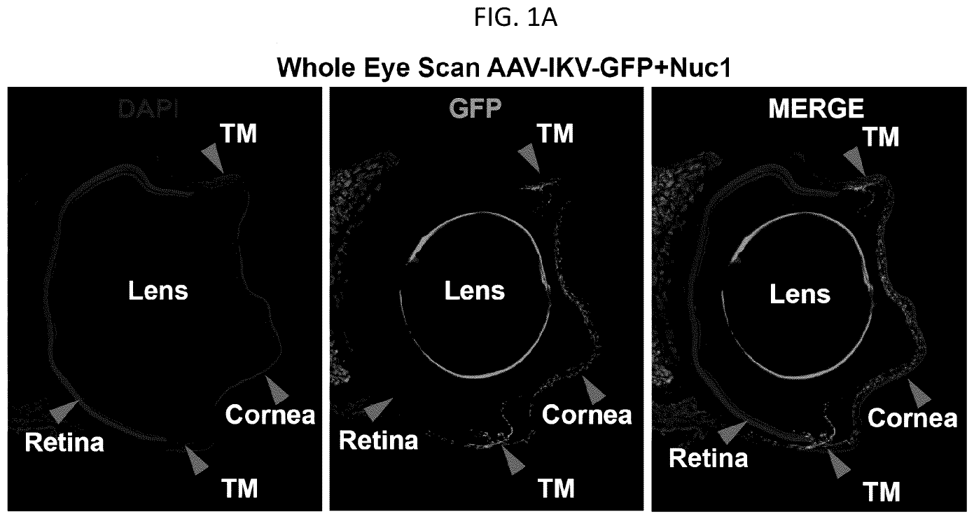

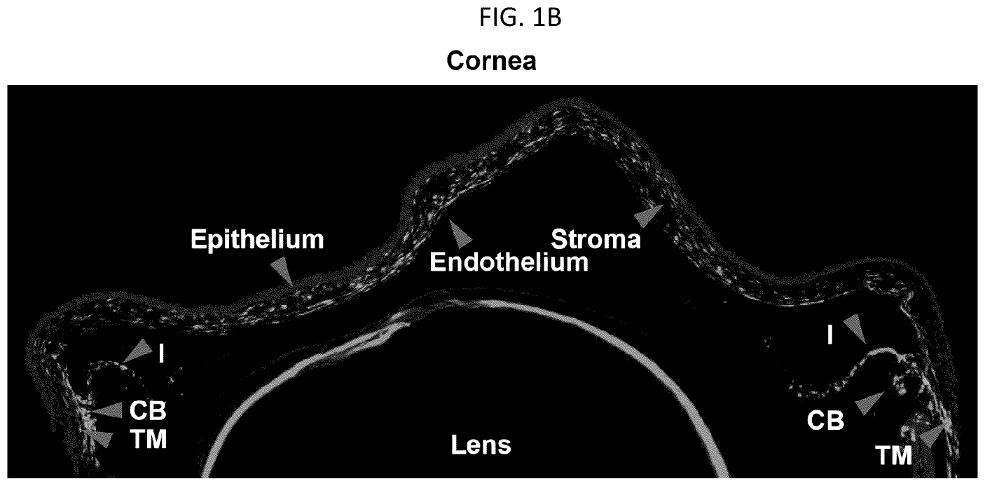

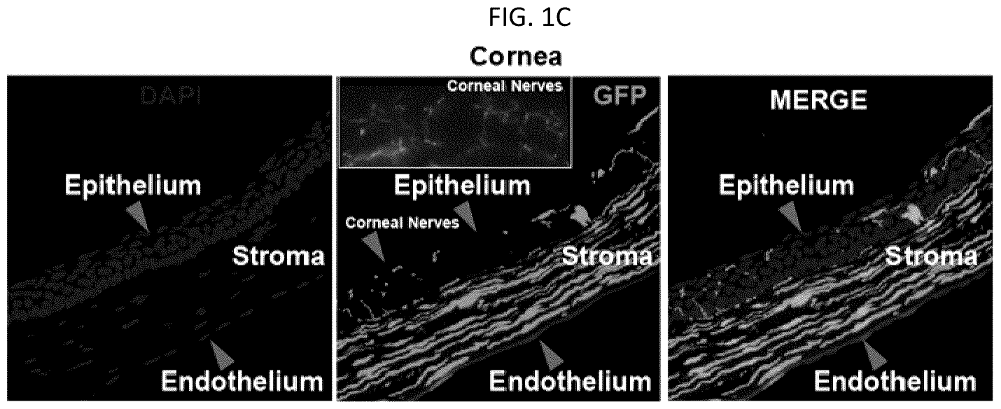

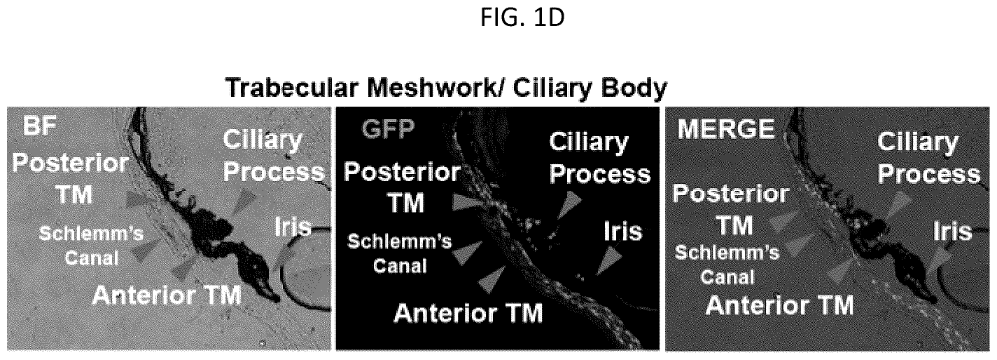

Dependent upon the serotype, intracamerally injected AAV infects primarily the corneal endothelium, stroma, or trabecular meshwork (33). Recently, AAV-IKV was shown to potently infect outer retinal cells following intravitreal injection (30), and AAV-IKV mediated infection of cells could be further enhanced when co-injected with a novel cell-penetrating peptide referred to as Nuc1 (31). To examine whether this same AAV-IKV/Nuc1 combination can infect cells in the anterior chamber, 1.1×10⁹ genome copies of a GFP-expressing AAV-IKV regulated by the chicken β actin promoter (AAV-IKV-GFP) and 1 μg Nuc1 were injected intracamerally into adult C57BL/6J mice. Two weeks following injection, frozen anterior chamber sections were examined for expression of GFP. Strongly GFP-positive cells were detected in the ciliary body, iris, cornea, and lens (FIG. 1A). Closer examination revealed that GFP expression was in the corneal stroma, ciliary body, and trabecular meshwork and some limited GFP expression was also observed in the corneal endothelium (FIG. 1B). Unexpectedly, the presence of GFP was also documented in some of the corneal nerves (FIG. 1C). The pigment in the ciliary process obscured some of the GFP expression. Bright field images merged with GFP signal revealed punctate GFP in the pigmented ciliary process and iris, suggesting that there is potentially greater GFP expression in these tissues than could be readily detected via fluorescence (FIG. 1D). These data demonstrate that the AAV-IKV/Nuc1 combination enables transgene delivery to a variety of cell types and locations in the anterior chamber of mice.

### TGFβ2 Mediated Increase in Intraocular Pressure

To investigate whether elevated TGFβ2 can increase intraocular pressure (IOP), a recombinant AAV-IKV expressing human TGFβ2 regulated by a chicken β actin promoter was constructed. The TGFβ2 cDNA was mutated to encode a serine instead of a cysteine at positions 226 and 228 in the TGFβ2 propeptide (NP_003229). This construct is referred to as AAV-IKV-TGFβ2^{CS}. These two mutations in TGFβ2 have been previously shown to result in a spontaneously active TGFβ2. Either 1.47×10⁹ genome units of AAV-IKV-TGFβ2^{CS} with 0.5 μg Nuc1 or a control vector without a cDNA (AAV-IKV-pA) was injected intracamerally and TGFβ2 expression was measured in frozen anterior chamber sections by immunohistochemistry one week after injection. Results show that, relative to uninjected animals, AAV-IKV-TGFβ2^{CS} injected animals expressed significantly greater levels of TGFβ2 in the ciliary body and trabecular meshwork (FIG. 2A). A higher level of TGFβ2 in the ciliary body of AAV-IKV-pA injected animals was also noted relative to uninjected animals, but these levels were significantly lower than that found in AAV-IKV-TGFβ2^{CS} injected animals, suggesting that some native TGFβ2 was upregulated as a consequence of either the AAV backbone or the injection procedure itself (FIG. 2A). Thus, AAV-IKV-TGFβ2^{CS} results in elevated expression of TGFβ2^{CS} in the ciliary body and trabecular meshwork when injected intracamerally in mice.

Having confirmed that AAV-IKV-TGFβ2^{CS} could be used to induce expression of TGFβ2^{CS} in the ciliary body and trabecular meshwork, whether elevated TGFβ2^{CS} would result in increased IOP was next investigated. 0.52×10⁹ vector genomes of AAV-IKV-TGFβ2^{CS} and 0.5 μg Nuc1 peptide (hereafter, except where indicated, all viral injections contained 0.5 μg Nuc1) were co-injected intracamerally into adult C57BL/6J mice. As a negative control, AAV-IKV-pA was injected in a separate group of animals. Following injection, intraocular pressure (IOP) in injected mice was measured on a weekly basis, specifically, at days 8, 16, 23, 30, 37, and 44 post injection. Results show that, at all timepoints examined, there was a statistically significant increase in IOP in AAV-IKV-TGFβ2^{CS} injected eyes relative to AAV-IKV-pA injected eyes (FIG. 2B; p≤0.0001, D8; p=0.0095, D16; p≤0.0001, D23; p≤0.0001, D30; p=0.03, D37; p=0.001, D44). IOP increase was rapid, detectable within one week after injection of AAV-IKV-TGFβ2^{CS}. Thus, AAV-IKV-TGFβ2^{CS} injected mice can be used as a model for testing inhibitors of TGFβ2^{CS}-mediated elevated IOP.

In a separate group of animals, the resulting IOP upon intracameral injection of a 2× dose of AAV-IKV-TGFβ2^{CS} was examined, specifically, 1.04×10⁹ vector genomes. As anticipated, there was also a statistically significant increase in IOP in this group of animals relative to AAV-IKV-pA injected animals at all time points, and the difference was greater than that seen with 0.52×10⁹ AAV-IKV-TGFβ2^{CS} vector genomes (FIG. 2B). However, when comparing the 1.04×10⁹ vector genome dose with the 0.52×10⁹ vector genome dose, a statistically significant increase was found in IOP at the higher dose at 3 of the 5 time points (p=0.12, D8; p≤0.0001, D16; p=0.03, D23; p=0.03, D30; p=0.24, D37; p=0.8866, D44).

Furthermore, intracameral injection of AAV-IKV-pA did not result in a statistically significant change in IOP relative to uninjected animals at any time point (see below). Thus, intracameral injection of AAV-IKV-TGFβ2^{CS} results in a significant increase in IOP in a dose dependent manner, and this increase is due to the expression of TGFβ2^{CS} and not the injection procedure or the recombinant AAV per se. Furthermore, the elevated IOP persists until at least day 44 post intracameral injection—the latest time point examined in this study.

### TGFβ2^{CS} Induces Fibrosis at the Trabecular Meshwork

Extracellular matrix deposition at the trabecular meshwork is believed to be a major cause of elevated IOP in glaucoma patients. To examine whether AAV-IKV-TGFβ2^{CS} injected animals exhibited any significant fibrosis of the trabecular meshwork, frozen sections from AAV-IKV-TGFβ2^{CS} injected animals were stained for α-smooth muscle actin (α-SMA) at 44 days post injection. Staining for α-SMA was found to be significantly elevated at the trabecular meshwork (FIG. 3A). Staining for α-SMA was undetectable under the same conditions in uninjected animals (FIG. 3B). However, some low levels of α-SMA staining was also detected in AAV-IKV-pA injected animals relative to uninjected animals (FIGS. 3C-3D), suggesting that either the injection procedure or AAV itself can contribute to some increase in α-SMA staining. However, the level of α-SMA staining associated with AAV-IKV-pA was substantially less than that associated with AAV-IKV-TGFβ2^{CS}. Thus, significantly elevated α-SMA staining in mice injected with AAV-IKV-TGFβ2^{CS} was largely associated with expression of TGFβ2^{CS}.

Expression of fibronectin was also examined in these studies. Results show that, similar to α-SMA, there were elevated levels of fibronectin at the trabecular meshwork in AAV-IKV-TGFβ2^{CS} injected animals relative to uninjected animals. However, like α-SMA, there was a higher level of fibronectin staining in AAV-IKV-pA injected animals relative to uninjected animals (FIG. 3D). Again, we conclude that the significantly elevated fibronectin staining in mice injected with

AAV-IKV-TGFβ2^{CS} was Largely Associated with Expression of TGFβ2^{CS}.

AAV-IKV mediated delivery of decorin inhibits AAV-IKV-TGFβ2C5 induced IOP Decorin is a small leucine rich proteoglycan that is expressed in the eye and is known to be able to bind and sequester TGFβ2. To examine whether human decorin could be expressed in the murine anterior segment, 0.52×10⁹ vector genomes of AAV-IKV-Decorin or AAV-IKV-pA was injected intracamerally into six-week-old C57BL/6J mice and frozen sections collected 7 days post injection were stained for decorin. Results show that, while there was a detectable level of decorin in AAV-IKV-pA injected eyes in the ciliary body, there was a significantly higher level of decorin expression in AAV-IKV-Decorin injected eyes (FIG. 4A).

Whether expression of decorin could inhibit an AAV-IKV-TGFβ2^{CS} induced increase in IOP in mice was next examined. 0.52×10⁹ vector genomes of AAV-IKV-TGFβ2^{CS} were injected intracamerally into adult C57BL/6J mice, either alone or with an equivalent dose of AAV-IKV-Decorin. Results show that, while there was no statistically significant difference in IOP between the AAV-IKV-TGFβ2^{CS} and AAV-IKV-TGFβ2^{CS}+AAV-IKV-Decorin groups initially, i.e., at day 8 (p=0.13) and day 16 (p=0.7), there was sustained lower IOP in AAV-IKV-TGFβ2^{CS}+AAV-IKV-Decorin injected group relative to the AAV-IKV-TGFβ2^{CS} injected group at all subsequent time points examined (p≤0.0001, D23; p≤0.0001, D30; p=0.016, D37; p=0.005, D44). Thus, expression of decorin results in a lowering of IOP in AAV-IKV-TGFβ2^{CS} animals. Such reduction in IOP takes approximately two weeks to be statistically significantly different from AAV-IKV-TGFβ2^{CS} injected animals. However, the reduction in IOP was sustained until day 44, the latest time point examined in this study. Furthermore, IOP in animals injected with AAV-IKV-TGFβ2^{CS}+AAV-IKV-Decorin was statistically indistinguishable from IOP in uninjected animals (FIG. 4B), suggesting that after the initial two weeks, IOP was reduced and maintained at normal levels.

### Decorin Inhibits TGFβ2^{CS} Induced Fibrosis at the Trabecular Meshwork

Above, intracameral injection of AAV-IKV-TGFβ2^{CS} with Nuc1 resulted in intense staining for α-SMA at the trabecular meshwork and in the ciliary body. Whether co-injection of AAV-IKV-Decorin with AAV-IKV-TGFβ2^{CS} would result in a reduced amount of fibrosis was examined. Results show that α-SMA as well as fibronectin staining was reduced at the trabecular meshwork in the presence of decorin relative to both the AAV-IKV-TGFβ2 injected groups (FIG. 5) as well as AAV-IKV-pA groups (FIGS. 3C-3D). We conclude that AAV-IKV mediated decorin can significantly inhibit TGFβ2^{CS} mediated fibrosis at the trabecular meshwork.

### Decorin Inhibits IOP Mediated Retinal Ganglion Cell Degeneration

The cardinal feature of glaucoma is retinal ganglion cell (RGC) degeneration, which is a consequence of elevated IOP. Loss of these cells is essentially the end stage of the disease and the major factor contributing to visual impairment in glaucoma patients. With the knowledge that AAV-IKV-Decorin counteracts the activity of TGFβ2^{CS}, it was expected that there would be a reduction in RGC death in AAV-IKV-TGFβ2^{CS} injected animals that were co-injected with AAV-IKV-Decorin. Thus, RGCs were stained with BRN3a in frozen sections from AAV-IKV-TGFβ2^{CS} and AAV-IKV-TGFβ2^{CS}+AAV-IKV-Decorin injected animals as well as uninjected or AAV-IKV-pA injected animals. Results show that there was no statistically significant difference in BRN3a staining between uninjected and AAV-IKV-pA injected animals. However, there was a statistically significant greater number of RGCs in the AAV-IKV-TGFβ2^{CS}+AAV-IKV-Decorin injected group relative to AAV-IKV-TGFβ2^{CS} injected animals (p=0.0322) (FIGS. 6A-6B). Thus, expression of decorin can inhibit TGFβ2^{CS} mediated death of RGC.

### AAV-IKV Mediated Gene Delivery to the Non-Human Primate Cornea

The results above suggest that use of AAV-IKV-Decorin for the treatment of glaucoma may be worth evaluating in a larger species of animal prior to consideration of testing in humans. Although conducting the studies described above in non-human primates (NHPs) is beyond the scope of the current study, as a critical first step, whether AAV-IKV-GFP is capable of transducing the NHP cornea via intracameral injection was investigated. Whether a non-GMP grade AAV-IKV-GFP and Nuc1 could be tolerated in NHPs was also investigated.

A total of 1.225×10¹¹ vector genomes of AAV-IKV-GFP and 12.5 μg Nuc1 were injected intracamerally in two eyes of one African green monkey (Chlorocebus sabaeus), which weighed 4.84 Kg and was approximately 9 to 11 years old. Color and fluorescence anterior and posterior photos were acquired 7 days prior to injection (injection defined as day 0, baseline) and at day 14 and 37 post injection. Additional parameters that were measured at day 14 and 37 included slit lamp, IOP and OCT. Body weights were measured 7 days prior to injection as well as day of injection and 4 days prior to end of study (day 41).

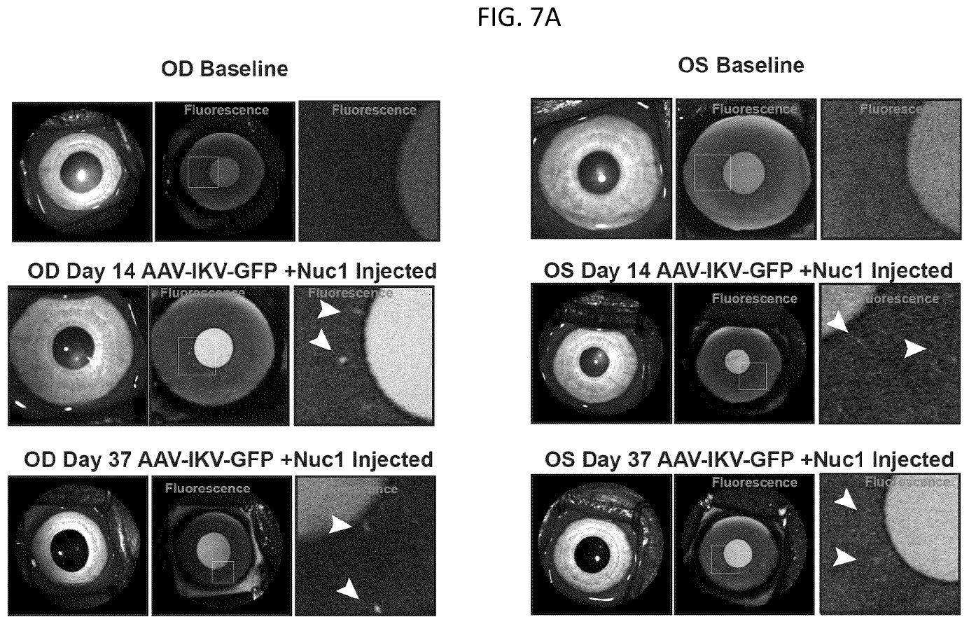

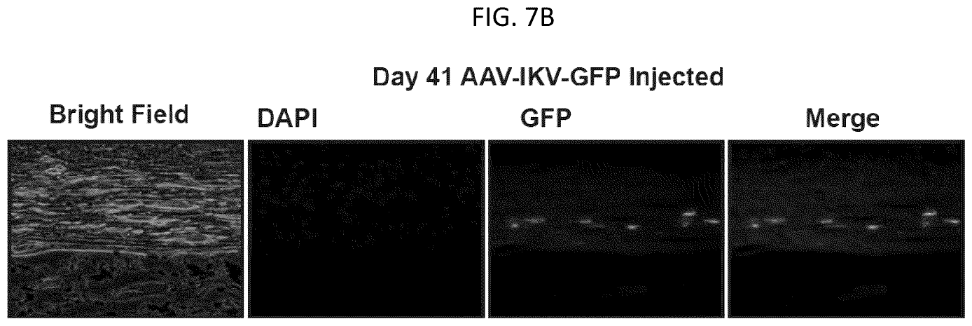

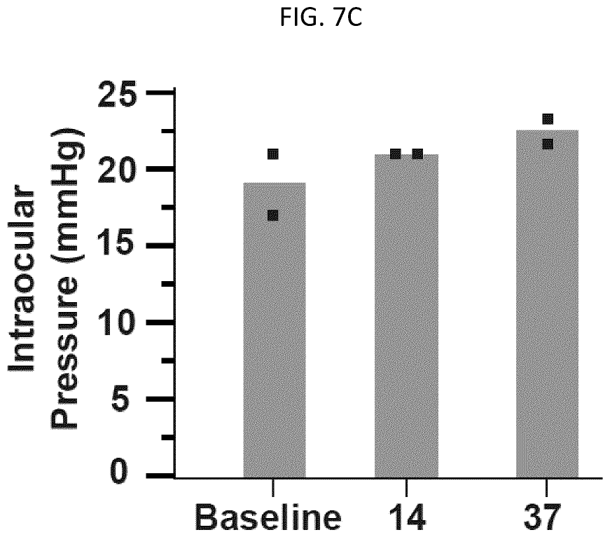

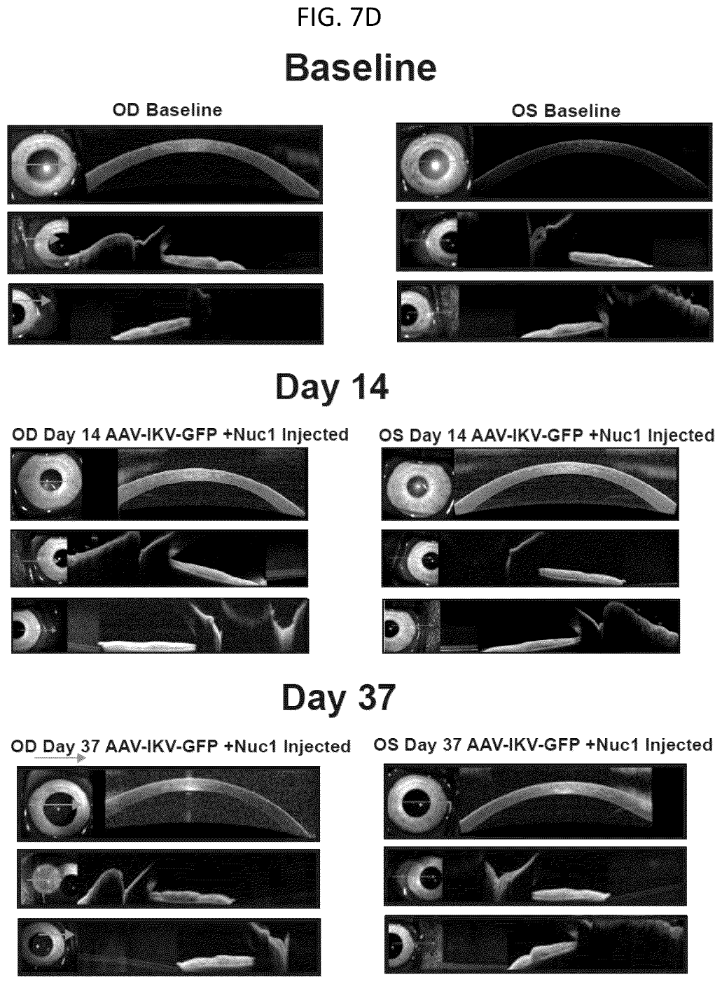

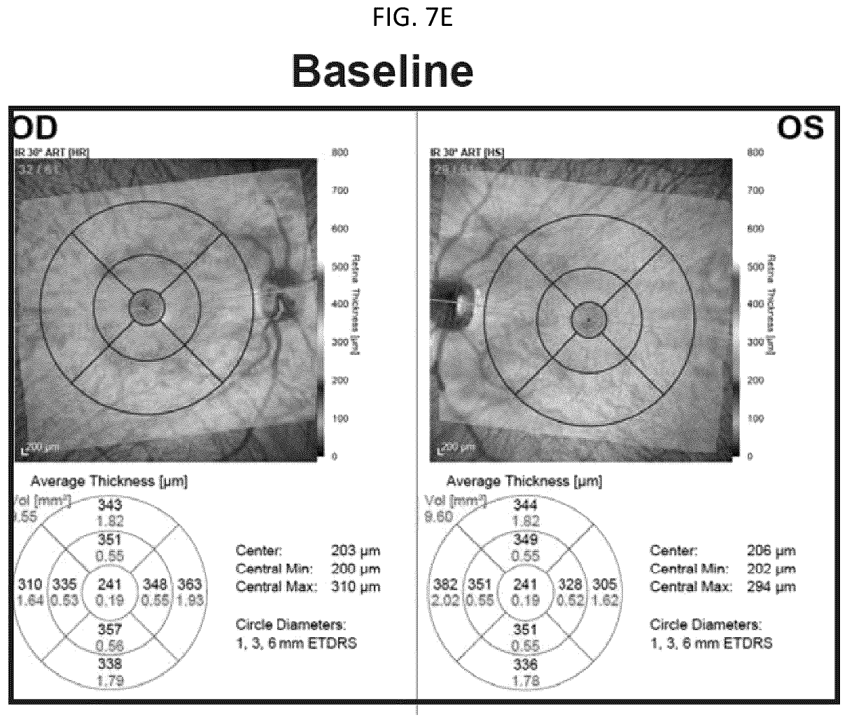

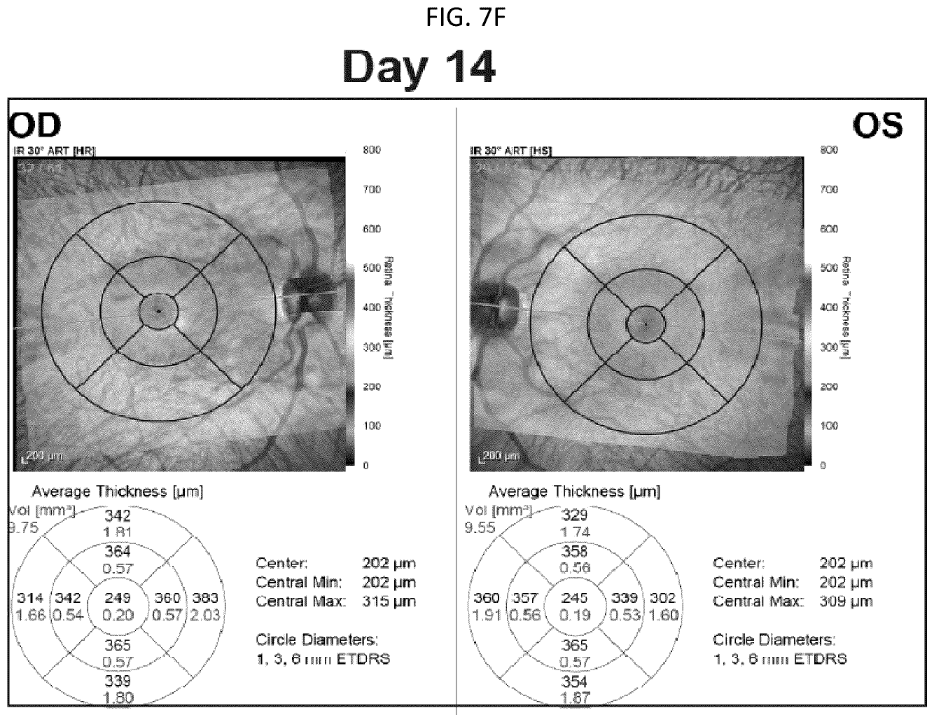

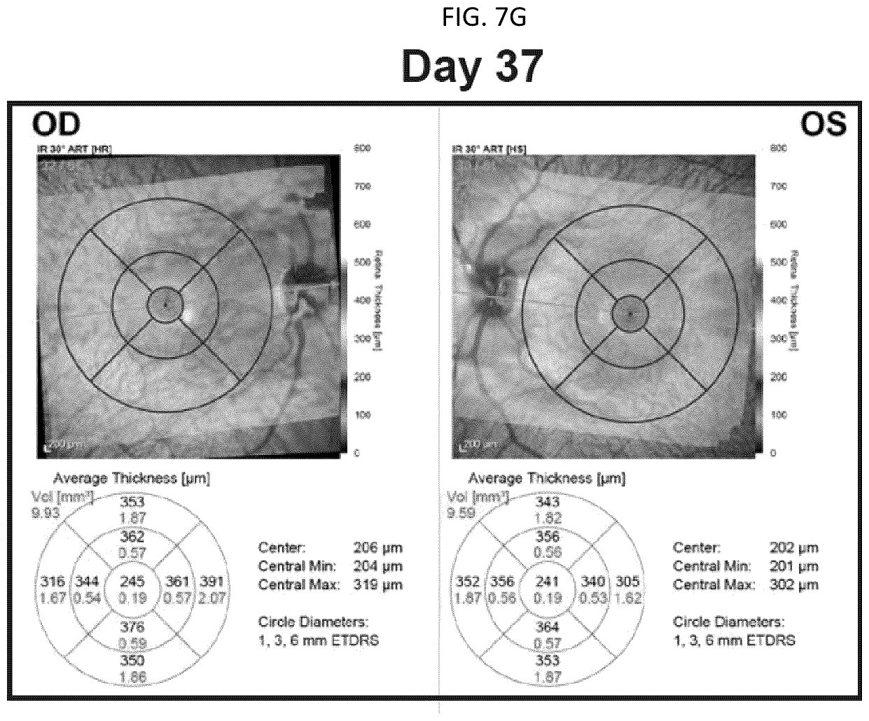

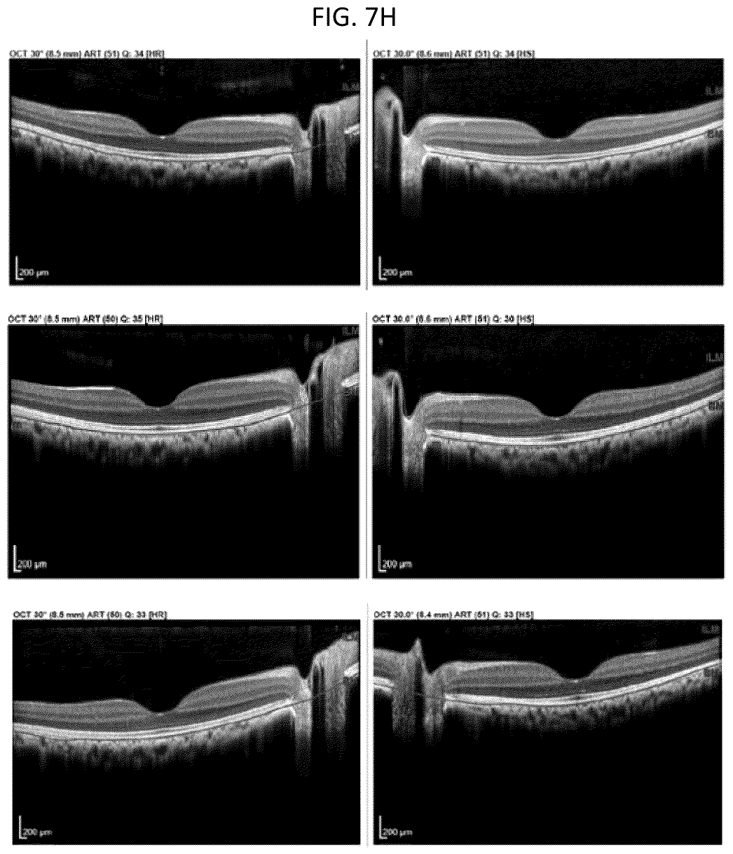

As anticipated, there was no detectable GFP fluorescence noted in either the oculus dexter (OD, right eye) or oculus sinister (OS, left eye) at baseline (FIG. 7A). However, at day 14 and day 37, punctate GFP fluorescence in live animals could be readily observed in both oculus dexter (OD, right eye) and oculus sinister (OS, left eye) (FIG. 7A arrowheads). Frozen sections collected at day 41 and examined by fluorescence microscopy revealed significant levels of GFP in the cornea (FIG. 7B). The IOP in OS and OD in AAV-IKV-GFP injected eyes did not differ significantly from baseline (FIG. 7C).

High resolution OCT imaging of cornea, sclera and anterior chamber angles of OS and OD revealed that AAV-IKV-GFP injected eyes at day 14 (middle) and day 37 (bottom) were similar to baseline (top) (FIG. 7D). Volume scans of OS and OD macula revealed an average thickness of the retina at day 14 (FIG. 7F) and 37 (FIG. 7G) was similar to baseline (FIG. 7E). High resolution OCT imaging of OS and OD retina of AAV-IKV-GFP injected eyes at day 14 (middle) and day 37 (bottom) was also similar to baseline (top) (FIG. 7H).

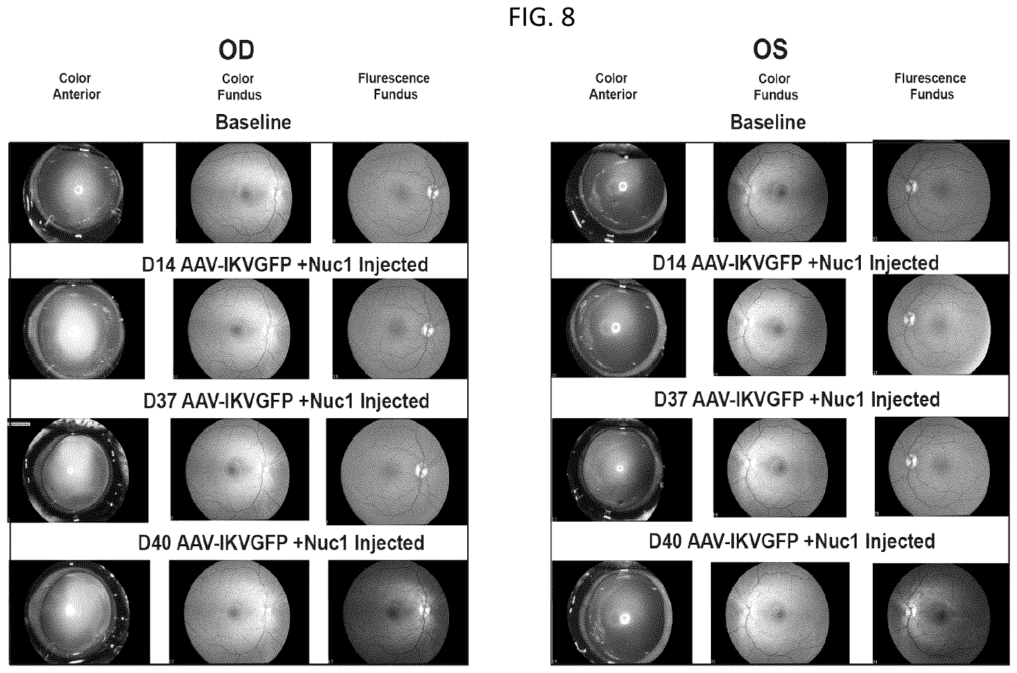

Anterior OS and OD fundus color as well as fluorescence fundus at day 14 and 37 were similar to baseline (FIG. 8). However, at day 40, there was some evidence of GFP expression in the fundus relative to baseline (FIG. 8). Finally, in AAV-IKV-GFP injected eyes the total clinical score (zero) comprising of but not limited to vitreous haze, retinal vasculitis, vitreous cell, lens opacity, anterior and posterior lens capsule deposit, iris exfoliation and synechia, aqueous flare, melanocytes, macrophages, corneal opacity, corneal vascular pannus and conjunctival congestion, swelling and discharge were all similar to baseline. In conclusion, our studies suggest that AAV-IKV-GFP coupled with Nuc1 is well tolerated in NHPs.

### DISCUSSION

In this study, the inventor has shown that AAV-IKV is a potent gene delivery vector for the anterior segment of the eye, including the ciliary body, cornea, and trabecular meshwork. This AAV vector can be used to increase the levels of constitutively active TGFβ2 in the murine eye, creating a model of glaucoma that exhibits increased IOP, fibrosis of the trabecular meshwork, and RGC death. Each of these parameters are key in the modeling of glaucoma. AAV-IKV mediated expression of decorin can significantly inhibit each of these outcomes of elevated expression of TGFβ2.

Several investigators have previously shown that intracameral injection of AAV expressing GFP can lead to transduction of corneal endothelium, stroma, and/or trabecular meshwork (33-37). In general, it was found that self-complementary AAV vectors performed significantly better than single-stranded genomes (38). However, in the disclosed studies, a single-stranded AAV vector was used. While self-complementary AAV genomes bypass second strand synthesis to enhance transgene expression, they have a limited capacity (approximately 2.2 Kb instead of 4.8 Kb) for a transgene expression cassette (39) (40).

Surprisingly, it was noted that intracameral injection of AAV-IKV could lead to GFP-positive corneal nerves. Corneal nerves have not been previously shown to be infected by any serotype of AAV despite these nerves being a significant target for gene therapy. The cornea is one of the most densely innervated tissues of the body, containing primarily sensory innervation (41). Corneal nerves form a subbasal plexus underneath the epithelium, which extends thin nerve leashes containing nociceptors present at the surface of the cornea that could be observed in our studies (42). These nerves are responsible for the sensations of touch, pain, and temperature. The trophic properties of corneal nerves are necessary to maintain a healthy ocular surface. Disruption of the corneal nerves has been found to impair corneal healing (43). Furthermore, diseases such as herpetic viral infections and ophthalmic surgical procedures cause corneal nerve disruption that can cause a range of conditions from mild dry eye to severe neurotrophic keratitis with corneal melting (44). While not a focus of this study, AAV-IKV may be useful for the delivery of genes to corneal nerves, a field that has not yet been explored.

TGFβ2 is produced as a latent 390-amino acid precursor that forms dimers through association of its N-terminal latency-associated peptide (LAP). The dimeric, mature, biologically active C-terminal 112-amino acid TGFβ2 is generated by the cleavage and dissociation of LAP. However, mutations introduced into the LAP domain result in a constitutively active TGFβ2 peptide. Previously, it has been shown that constitutively active TGFβ2 expressed from an adenovirus vector injected intracamerally into rodents leads to an increase in IOP (26). However, adenovirus vectors are known to cause inflammation that ultimately causes a significant reduction of transgene expression. The immune responses associated with adenovirus are likely to significantly confound the phenotypic impact from elevated expression of TGFβ2. For these and additional reasons, AAV vectors were used in these disclosed studies, notwithstanding that Nuc1 peptide may be immunogenic, but no significant inflammation in the AAV-injected mice and NHP was noted in these studies. Indeed, AAV has been consistently described as a relatively benign vector that does not cause significant inflammation in the eye relative to adenovirus. However, since an empty AAV vector (i.e., AAV-IKV-pA) also led to some mild fibrosis and TGFβ2 expression, AAV was not completely benign in the context of the disclosed studies. However, the levels of elevated TGFβ2 and fibrosis from AAV-IKV were not significant enough to cause an increase in IOP. Moreover, in these studies research grade AAV vectors were used, and whether similar outcomes are documented using GMP grade vectors remains to be seen in future studies. In the context of clinical use, only the safety of AAV-IKV-Decorin and Nuc1 would be relevant.

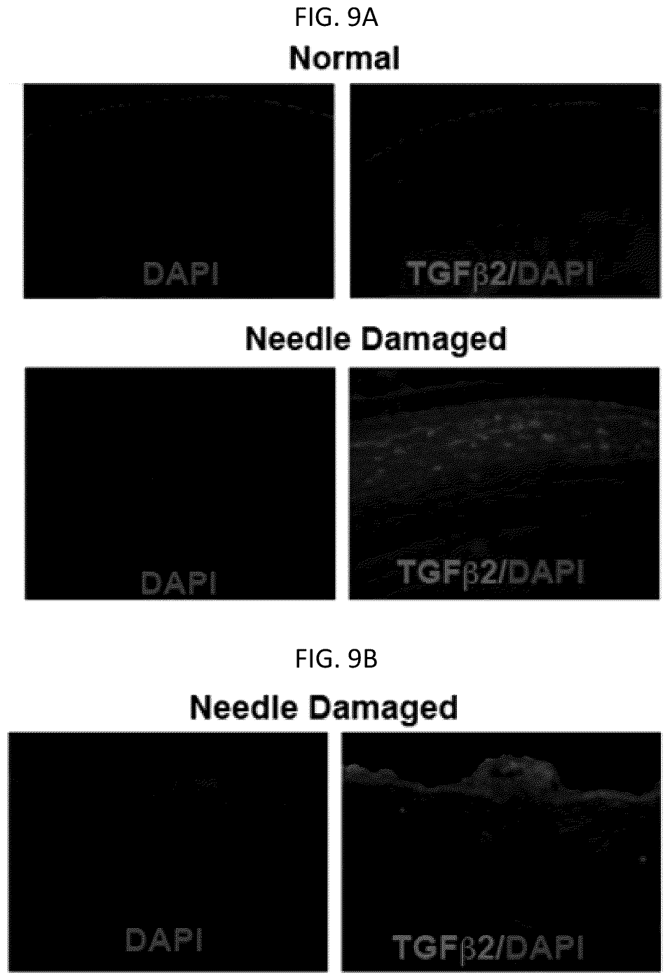

Relative to humans, the murine lens occupies a very large volume of the eye. Consequently, intravitreal injections in mice sometimes result in damage to the lens epithelium. Incidentally, we noticed that, in such animals, there was a significant elevation of TGFβ2 in the lens epithelial cells (FIG. 9A). In some animals, the lens epithelial cells form a mass of cells on the surface of the lens (FIG. 9B), and it was expected that such (loose) cells may migrate to the trabecular meshwork following lensectomy in IAG, resulting in fibrosis at the trabecular meshwork and increased IOP, as discussed in detail previously (21). In comparison to humans, the mouse lens occupies a very large volume of the eye and thus performing a lensectomy in mice to model IAG is technically challenging.

Onset of transgene expression from AAV-IKV was rapid, with IOP increasing in AAV-IKV-TGFβ2^{CS} injected animals within days. Elevated IOP was maintained out to day 44 post-injection, the latest time point examined in this study. The increase in IOP was dependent upon the dose of AAV-IKV-TGFβ2^{CS} injected into animals.

Although generation of an animal model of glaucoma in NHPs is beyond the scope of the current study, evidence that AAV-IKV-GFP can infect NHP cornea following intracameral injection was also provided. Clear evidence was found of GFP fluorescence in live animals as well as in cryosections in AAV-IKV-GFP injected eyes at day 14 and day 37 post injection, the latest time point examined. At all timepoints, the cornea of both eyes exhibited transparency similar to baseline without any sign of inflammation, suggesting that the research grade vector did not lead to any significant immune responses. Furthermore, the IOP in AAV-IKV-GFP injected eyes did not differ significantly from baseline, and anterior OCT images of AAV-IKV-GFP injected eyes were similar to baseline at all timepoints. Finally, volume scans of macula, blue laser autofluorescence wide-field images, and all other clinical parameters in AAV-IKV-GFP injected eyes were found to be similar to baseline, indicating that AAV-IKV-GFP satisfies the initial criteria for further investigation as a candidate AAV for development of a gene therapy for glaucoma.

One limitation of our study is that while AAV-IKV-Decorin could inhibit AAV-IKV-TGFb2^{CS} increase in IOP, fibrosis and ganglion cell death, our studies do not address whether pre-established IOP and fibrosis can be reversed. In order to conduct such a study, one would need to conduct multiple injections at different time points in the mouse eye. Such studies are technically challenging due to the small eyes of mice. Alternatively, one may develop an AAV-IKV-TGFb2^{CS} where the transgene can be turned off after the observation of pathology and such mice subsequently injected with AAV-IKV-Decorin. Such studies are contemplated but are beyond the scope of the present studies.

Decorin is naturally expressed in the eye in multiple tissues (23) and recombinant decorin has previously been injected into human eyes without any negative consequences (45), thus the approach described in this study provides a potential therapy for glaucoma.

### REFERENCES

ADDIN EN.REFLIST 1 Tham, Y. C. et al. Global prevalence of glaucoma and projections of glaucoma burden through 2040: a systematic review and meta-analysis. Ophthalmology 121, 2081-2090, doi: 10.1016/j.ophtha.2014.05.013 (2014).
2 Foster, A. & Gilbert, C. Cataract in children. Acta Paediatr 92, 1376-1378, doi: 10.1080/08035250310007556 (2003).
3 Khan, A. O. & Al-Dahmash, S. Age at the time of cataract surgery and relative risk for aphakic glaucoma in nontraumatic infantile cataract. J AAPOS 13, 166-169, doi: 10.1016/j.jaapos.2008.10.020 (2009).
4 Kountouras, J., Zavos, C. & Deretzi, G. Primary open-angle glaucoma. N Engl J Med 360, 2679; author reply 2679-2680 (2009).
5 Weinreb, R. N., Aung, T. & Medeiros, F. A. The pathophysiology and treatment of glaucoma: a review. JAMA 311, 1901-1911, doi: 10.1001/jama.2014.3192 (2014).
6 Kaufman, P. L., Mohr, M. E., Riccomini, S. P. & Rasmussen, C. A. Glaucoma Drugs in the Pipeline. Asia Pac J Ophthalmol (Phila) 7, 345-351, doi: 10.22608/APO.2018298 (2018).
7 Lusthaus, J. A. & Goldberg, I. Investigational and experimental drugs for intraocular pressure reduction in ocular hypertension and glaucoma. Expert Opin Investig Drugs 25, 1201-1208, doi: 10.1080/13543784.2016.1223042 (2016).
8 Gemenetzi, M., Yang, Y. & Lotery, A. J. Current concepts on primary open-angle glaucoma genetics: a contribution to disease pathophysiology and future treatment. Eye (Lond) 26, 355-369, doi: 10.1038/eye.2011.309 (2012).
9 Tripathi, R. C., Li, J., Chan, W. F. & Tripathi, B. J. Aqueous humor in glaucomatous eyes contains an increased level of TGF-beta 2. Exp Eye Res 59, 723-727, doi: 10.1006/exer.1994.1158 (1994).
10 Inatani, M. et al. Transforming growth factor-beta 2 levels in aqueous humor of glaucomatous eyes. Graefes Arch Clin Exp Ophthalmol 239, 109-113, doi: 10.1007/s004170000241 (2001).
11 Picht, G., Welge-Luessen, U., Grehn, F. & Lutjen-Drecoll, E. Transforming growth factor beta 2 levels in the aqueous humor in different types of glaucoma and the relation to filtering bleb development. Graefes Arch Clin Exp Ophthalmol 239, 199-207, doi: 10.1007/s004170000252 (2001).
12 Ochiai, Y. & Ochiai, H. Higher concentration of transforming growth factor-beta in aqueous humor of glaucomatous eyes and diabetic eyes. Jpn J Ophthalmol 46, 249-253, doi: 10.1016/s0021-5155 (01) 00523-8 (2002).
13 Ozcan, A. A., Ozdemir, N. & Canataroglu, A. The aqueous levels of TGF-beta2 in patients with glaucoma. Int Ophthalmol 25, 19-22, doi: 10.1023/b: inte.0000018524.48581.79 (2004).
14 NikhalaShree, S. et al. Lowered Decorin With Aberrant Extracellular Matrix Remodeling in Aqueous Humor and Tenon's Tissue From Primary Glaucoma Patients. Invest Ophthalmol Vis Sci 60, 4661-4669, doi: 10.1167/iovs.19-27091 (2019).
15 Min, S. H., Lee, T. I., Chung, Y. S. & Kim, H. K. Transforming growth factor-beta levels in human aqueous humor of glaucomatous, diabetic and uveitic eyes. Korean J Ophthalmol 20, 162-165, doi: 10.3341/kjo.2006.20.3.162 (2006).
16 Jampel, H. D., Roche, N., Stark, W. J. & Roberts, A. B. Transforming growth factor-beta in human aqueous humor. Curr Eye Res 9, 963-969, doi: 10.3109/02713689009069932 (1990).
17 Agarwal, P., Daher, A. M. & Agarwal, R. Aqueous humor TGF-beta2 levels in patients with open-angle glaucoma: A meta-analysis. Mol Vis 21, 612-620 (2015).
18 Sisto, M., Ribatti, D. & Lisi, S. Organ Fibrosis and Autoimmunity: The Role of Inflammation in TGFbeta-Dependent EMT. Biomolecules 11, doi: 10.3390/biom11020310 (2021).
19 Das, V., Bhattacharya, S., Chikkaputtaiah, C., Hazra, S. & Pal, M. The basics of epithelial-mesenchymal transition (EMT): A study from a structure, dynamics, and functional perspective. J Cell Physiol, doi: 10.1002/jcp.28160 (2019).
20 Michael, I., Walton, D. S. & Levenberg, S. Infantile aphakic glaucoma: a proposed etiologic role of IL-4 and VEGF. J Pediatr Ophthalmol Strabismus 48, 98-107, doi: 10.3928/01913913-20100518-04 (2011).
21 Yeung, H. H., Kumar-Singh, R. & Walton, D. S. Infantile Aphakic Glaucoma: A Proposed Mechanism. J Pediatr Ophthalmol Strabismus 59, 236-242, doi: 10.3928/01913913-20210929-02 (2022).
22 Neill, T., Schaefer, L. & Iozzo, R. V. Decorin: a guardian from the matrix. Am J Pathol 181, 380-387, doi: 10.1016/j.ajpath.2012.04.029 (2012).
23 Schneider, M. et al. A novel ocular function for decorin in the aqueous humor outflow. Matrix Biol, doi: 10.1016/j.matbio.2021.02.002 (2021).
24 Nikhalashree, S. et al. Anti-glaucoma medications lowered decorin and altered profibrotic proteins in human tenon's fibroblasts. Exp Eye Res 224, 109199, doi: 10.1016/j.exer.2022.109199 (2022).
25 Hill, L. J. et al. Decorin Reduces Intraocular Pressure and Retinal Ganglion Cell Loss in Rodents Through Fibrolysis of the Scarred Trabecular Meshwork. Invest Ophthalmol Vis Sci 56, 3743-3757, doi: 10.1167/iovs. 14-15622 (2015).
26 Shepard, A. R. et al. Adenoviral gene transfer of active human transforming growth factor-{beta} 2 elevates intraocular pressure and reduces outflow facility in rodent eyes. Invest Ophthalmol Vis Sci 51, 2067-2076, doi: 10.1167/iovs.09-4567 (2010).
27 Agrahari, V. et al. A comprehensive insight on ocular pharmacokinetics. Drug Deliv Transl Res 6, 735-754, doi: 10.1007/s13346-016-0339-2 (2016).
28 Vaajanen, A. & Vapaatalo, H. A Single Drop in the Eye-Effects on the Whole Body? Open Ophthalmol J 11, 305-314, doi: 10.2174/1874364101711010305 (2017).
29 Almasich, M. & Levin, L. A. Neuroprotection in Glaucoma: Animal Models and Clinical Trials. Annu Rev Vis Sci 3, 91-120, doi: 10.1146/annurev-vision-102016-061422 (2017).
30 Kumar, B., Mishra, M., Cashman, S. & Kumar-Singh, R. Retinal Penetrating Adeno-Associated Virus. Invest Ophthalmol Vis Sci In press, doi: 10.1167/iovs.65.8.31 (2024).
31 Kumar, B., Mishra, M., Talreja, D., Cashman, S. & Kumar-Singh, R. Cell-Penetrating Chaperone Nuc1 for Small- and Large-Molecule Delivery Into Retinal Cells and Tissues. Invest Ophthalmol Vis Sci 65, 31, doi: 10.1167/iovs.65.8.31 (2024).
32 Macda, A. et al. Circadian intraocular pressure rhythm is generated by clock genes. Invest Ophthalmol Vis Sci 47, 4050-4052, doi: 10.1167/iovs.06-0183 (2006).
33 Rodriguez-Estevez, L., Asokan, P. & Borras, T. Transduction optimization of AAV vectors for human gene therapy of glaucoma and their reversed cell entry characteristics. Gene Ther 27, 127-142, doi: 10.1038/s41434-019-0105-4 (2020).
34 Frederick, A. et al. Engineered Capsids for Efficient Gene Delivery to the Retina and Cornea. Hum Gene Ther 31, 756-774, doi: 10.1089/hum.2020.070 (2020).
35 Wang, L., Xiao, R., Andres-Mateos, E. & Vandenberghe, L. H. Single stranded adeno-associated virus achieves efficient gene transfer to anterior segment in the mouse eye. PLOS One 12, c0182473, doi: 10.1371/journal.pone.0182473 (2017).
36 Bogner, B. et al. Capsid Mutated Adeno-Associated Virus Delivered to the Anterior Chamber Results in Efficient Transduction of Trabecular Meshwork in Mouse and Rat. PLOS One 10, c0128759, doi: 10.1371/journal.pone.0128759 (2015).
37 Kong, F. et al. Self-complementary AAV5 vector facilitates quicker transgene expression in photoreceptor and retinal pigment epithelial cells of normal mouse. Exp Eye Res 90, 546-554, doi: 10.1016/j.exer.2010.01.011 (2010).
38 Buie, L. K. et al. Self-complementary AAV virus (scAAV) safe and long-term gene transfer in the trabecular meshwork of living rats and monkeys. Invest Ophthalmol Vis Sci 51, 236-248, doi: 10.1167/iovs.09-3847 (2010).
39 McCarty, D. M. Self-complementary AAV vectors; advances and applications. Mol Ther 16, 1648-1656, doi: 10.1038/mt.2008.171 (2008).
40 McCarty, D. M., Monahan, P. E. & Samulski, R. J. Self-complementary recombinant adeno-associated virus (scAAV) vectors promote efficient transduction independently of DNA synthesis. Gene Ther 8, 1248-1254, doi: 10.1038/sj.gt.3301514 (2001).
41 Marfurt, C. F. & Ellis, L. C. Immunohistochemical localization of tyrosine hydroxylase in corneal nerves. J Comp Neurol 336, 517-531, doi: 10.1002/cne.903360405 (1993).
42 Lazarov, N. E. Comparative analysis of the chemical neuroanatomy of the mammalian trigeminal ganglion and mesencephalic trigeminal nucleus. Prog Neurobiol 66, 19-59, doi: 10.1016/s0301-0082 (01) 00021-1 (2002).
43 Murphy, C. J. et al. Spontaneous chronic corneal epithelial defects (SCCED) in dogs: clinical features, innervation, and effect of topical SP, with or without IGF-1. Invest Ophthalmol Vis Sci 42, 2252-2261 (2001).
44 Wilson, S. E. & Ambrosio, R. Laser in situ keratomileusis-induced neurotrophic epitheliopathy. Am J Ophthalmol 132, 405-406, doi: 10.1016/s0002-9394 (01) 00995-3 (2001).
45 Abdullatif, A. M. et al. Intravitreal decorin preventing proliferative vitreoretinopathy in perforating injuries: a pilot study. Graefes Arch Clin Exp Ophthalmol 256, 2473-2481, doi: 10.1007/s00417-018-4105-7 (2018).

What is claimed:

1. A method of treating an eye disease in a subject, the method comprising administering a recombinant adeno-associated virus (AAV) vector to an eye of the subject, wherein the AAV vector comprises:
a) a viral capsid protein into which an IKV peptide comprising SEQ ID NO: 1 has been inserted, and
b) a polynucleotide comprising a promoter operably linked to a sequence encoding a decorin protein.

2. The method of claim 1, wherein the administration is intracameral.

3. The method of claim 1 further comprising administering a Nuc1 peptide to the subject, wherein the Nuc1 peptide comprises:
a) SEQ ID NO: 1 linked to SEQ ID NO: 2 via a flexible linker, or
b) SEQ ID NO: 3.

4. The method of claim 1, wherein the IKV peptide comprises SEQ ID NO: 6.

5. The method of claim 1, wherein the decorin protein comprises SEQ ID NO: 9.

6. The method of claim 1, wherein the AAV is serotype 2 AAV pseudotyped with an AAV 9 capsid (AAV2/9).

7. The method of claim 1, wherein the decorin protein is expressed in the anterior chamber of the eye of the subject following administration of the AAV vector.

8. The method of claim 7, wherein the decorin protein is expressed in the trabecular meshwork, ciliary body, corneal stroma, and/or corneal endothelium.

9. The method of claim 1, wherein, following administration of the AAV vector:
a) intraocular pressure (IOP) is reduced in the eye of the subject;
b) fibrosis at the trabecular meshwork is reduced in the eye of the subject; and/or
c) retinal ganglion cell death is reduced in the eye of the subject.

10. The method of claim 1, wherein the eye disease is glaucoma.

11. The method of claim 10, wherein the glaucoma is primary open angle glaucoma (POAG) or infantile aphakic glaucoma (IAG).

12. A method of generating an animal model of glaucoma by administering a recombinant AAV vector to an eye of an animal, wherein the AAV vector comprises:
a) a viral capsid protein into which an IKV peptide comprising SEQ ID NO: 1 has been inserted, and
b) a polynucleotide comprising a promoter operably linked to a sequence encoding a constitutively active variant of transforming growth factor beta 2 (TGFβ2^{CS}).

13. The method of claim 12, wherein the administration is intracameral.

14. The method of claim 12, wherein a Nuc1 peptide is co-administered with the recombinant AAV vector to an eye of the animal to generate the animal model, and wherein the Nuc1 peptide comprises:
a) SEQ ID NO: 1 linked to SEQ ID NO: 2 via a flexible linker, or
b) SEQ ID NO: 3.

15. The method of claim 12, wherein the IKV peptide comprises SEQ ID NO: 6.

16. The method of claim 12, wherein the TGFβ2^{CS} comprises SEQ ID NO: 7.

17. The method of claim 12, wherein the AAV is serotype 2 AAV pseudotyped with an AAV 9 capsid (AAV2/9).

18. The method of claim 12, wherein the TGFβ2^{CS} is expressed in the anterior chamber of the eye of the animal following administration of the AAV vector.

19. The method of claim 18, wherein the TGFβ2^{CS} is expressed in the ciliary body and trabecular meshwork.

20. The method of claim 12, wherein the animal model exhibits:
a) increased IOP relative to a control animal;
b) increased fibrosis at the trabecular meshwork relative to a control animal; and/or
c) increased retinal ganglion cell death relative to a control animal.
